(19) United States
(12) Patent Application Publication
(10) Pub. No.: US 2016/0263150 A1
(43) Pub. Date: Sep. 15, 2016

(54) TUNING BIOLOGICAL SYSTEM PROPERTIES FOR OPTIMIZED TREATMENT EFFICACY

(71) Applicants: Nadia C. Lara, Houston, TX (US); Andrew R. Barron, Houston, TX (US); Stuart Corr, Houston, TX (US); Steven Curley, Missouri City, TX (US)

(72) Inventors: Nadia C. Lara, Houston, TX (US); Andrew R. Barron, Houston, TX (US); Stuart Corr, Houston, TX (US); Steven Curley, Missouri City, TX (US)

(73) Assignees: Baylor College of Medicine, Houston, TX (US); William Marsh Rice University, Houston, TX (US)

(21) Appl. No.: 15/067,010

(22) Filed: Mar. 10, 2016

Related U.S. Application Data
(60) Provisional application No. 62/131,123, filed on Mar. 10, 2015.

(51) Int. Cl. A61K 33/00

(52) U.S. Cl. CPC A61K 33/00

(57) ABSTRACT

Various embodiments of the present disclosure pertain to methods of optimizing a treatment efficacy of a biological system by tuning a property of the biological system through the addition of an optimizing agent to the biological system. The tuning can include: (a) determining a property parameter of the biological system; (b) selecting an optimizing agent to be added to the biological system based on the determined property parameter; and (c) adding the optimizing agent to the biological system. The optimizing agent can include a kosmotropic material. The biological system can include a tissue, such as a tumor. The methods of the present disclosure can be utilized to enhance the efficacy of various treatments, such as the heat treatment of a biological system exposed to a radiofrequency field. The methods of the present disclosure can also include a step of treating the biological system.

### CROSS-REFERENCE TO RELATED APPLICATIONS

This application claims priority to U.S. Provisional Patent Application No. 62/131,123, filed on Mar. 10, 2015. The entirety of the aforementioned application is incorporated herein by reference.

### STATEMENT REGARDING FEDERALLY SPONSORED RESEARCH

This invention was made with government support under Grant No. 1450681, awarded by the National Science Foundation. The government has certain rights in the invention.

### BACKGROUND

Current methods of treating various biological systems have numerous limitations, including limited treatment efficacies and the need for complex drugs. Various aspects of the present disclosure address such limitations.

### SUMMARY

In some embodiments, the present disclosure pertains to methods of optimizing a treatment efficacy of a biological system. In some embodiments, the methods of the present disclosure include a step of tuning a property of the biological system by adding an optimizing agent to the biological system. In some embodiments, the tuning includes the following steps: (a) determining a property parameter of the biological system; (b) selecting an optimizing agent to be added to the biological system based on the determined property parameter; and (c) adding the optimizing agent to the biological system.

In some embodiments, the optimizing agent includes a kosmotropic material, such as alcohols, zwitterionic molecules, amines, neutral molecules, water soluble molecules, water soluble nanomaterials, carbohydrates, amino acids, organosulfur compounds, and combinations thereof. In some embodiments, the tuned property includes the heating rate of the biological system. In some embodiments, the added optimizing agent increases the heating rate of the biological system.

In some embodiments, the biological system includes, without limitation, aqueous biological systems, body fluids, blood, solid biological systems, tissues, organs, vasculatures, matrices, and combinations thereof. In some embodiments, the biological system is a tissue, such as a tumor. In some embodiments, the biological system is part of a subject or isolated from the subject. In some embodiments, the subject is a human being suffering from a condition, such as cancer.

The methods of the present disclosure can be utilized to enhance the efficacy of various treatments. For instance, in some embodiments, the optimized treatment includes radiation treatment of a biological system. In some embodiments, the optimized treatment includes heat treatment (e.g., hyperthermia treatment) of a biological system exposed to a radiofrequency field.

In some embodiments, the methods of the present disclosure can be utilized to tune the property of a biological system in a selective manner. For instance, in some embodiments the property of a first biological system is tuned while the property of a second biological system remains substantially the same. In some embodiments, the first biological system is associated with a condition while the second biological system is not associated with the condition. In some embodiments, the first biological system is a tumor, and the second biological system is normal tissue.

In additional embodiments, the methods of the present disclosure also include a step of treating the biological system. In some embodiments, the treating includes exposing the biological system to heat (e.g., heat as a result of the interaction of the biological system with a radiofrequency field).

### DESCRIPTION OF THE FIGURES

FIG. 1 provides a scheme of a method of optimizing the treatment efficacy of a biological system.

FIGS. 2A-B provide an experimental setup for the radiofrequency (RF)-induced heating of aqueous solutions. FIG. 2A shows an image of a radiofrequency (13.56 MHz) generator (right arrow) that is used to generate a focused, high-voltage electric field between the transmitting (Tx) and receiving (Rx) heads. An infrared camera (left arrow) is used to record the temperature of the sample (center arrow). FIG. 2B provides temperature plots for 0.1, 1, and 10 mM NaCl solutions. Least-squares linear regressions (solid lines) are used to calculate RF heating rates. Insert displays the IR camera view of the sample.

FIGS. 3A-B provide data relating to the RF heating of salt solutions. FIG. 3A provides a concentration-dependent RF heating at 100 W generator power of monovalent (NaCl—▪, NaC₂H₃O₂—□), divalent (MgCl₂—▴, Na₂SO₄—Δ), and trivalent salts (AuCl₃—, Na₃C₆H₅O₇—∘). Solid lines indicate Gaussian fit to the corresponding valency. FIG. 3B provides a conductivity (at 13.56 MHz) dependence of RF heating rates for all aqueous salt solutions (▪). Complex aqueous systems (empty symbols) heat similarly to aqueous salt solutions of similar conductivity. All heating rates and conductivity values reported are averages of three replicates with error bars indicating SD. Error bars may be smaller than symbols.

FIGS. 4A-B provide data relating to the characterization of single-walled carbon nanotubes (SWNTs). FIG. 4A provides data relating to the radial breathing mode region of the Raman spectra for SWNTs, which show the distribution of chiralities within the samples. FIG. 4B provides x-ray photoelectron spectroscopy (XPS) of purified samples, which show minimal residual catalyst content.

FIGS. 5A-C provide illustrations regarding the modulation of the conductivity and heating of phosphate buffered saline (PBS) and blood. FIG. 5A provides chemical structures of kosmotropic materials used for RF heating enhancement. FIG. 5B shows that kosmotropic materials enhance the heating rate of PBS () by reducing its conductivity. Materials used include sugar alcohols (red; ethylene glycol—□, propylene glycol—∘, glycerol—Δ, sorbitol—∇), sugars (blue; maltose—□, glucose—∘, sucrose—Δ), amines (black; glycine—□, sarcosine—∘, glycine betaine—Δ), and dimethylsulfoxide (purple, □). FIG. 5C shows that the conductivity and heating rate of blood (▪) are modulated in a concentration dependent manner by propylene glycol (□), glycerol (∘), water (Δ) and normal saline (∘). Arrows indicate increasing additive content. Dashed lines in FIGS. 5B-C indicate a general salt heating curve trend. All heating rates and conductivity values reported are averages of three replicates with error bars indicating standard deviation (SD). Error bars in FIG. 5C are omitted for clarity but are generally smaller than the symbols.

FIGS. 6A-B provide data relating to the RF heating of nanoparticle suspensions. FIG. 6A provides illustrations of nanoparticles used in RF experiments, including SWNTs, tannic acid-functionalized gold nanoparticles (t-AuNPs), and citrate-functionalized gold nanoparticles (c-AuNPs). FIG. 6B provides conductivity dependent heating rates of nanoparticle suspensions (c-AuNPs, t-AuNPs, and SWNTs) compared to NaCl solutions under RF exposure at 900 W generator power. All heating rates and conductivity values reported are averages of three replicates with error bars indicating SD. The error bars may be smaller than symbols.

FIGS. 7A-C provide data relating to the supernatant heating of AuNP and SWNT suspensions. RF-induced heating rates are shown for c-AuNPs at 500 mg/L (FIG. 7A), t-AuNPs at 1000 mg/L (FIG. 7B), and SWNTs at 200 mg/L (FIG. 7C). Solid bars represent the nanoparticle suspension heating rates and hatched bars represent corresponding supernatant heating rates following centrifugation.

FIG. 8A-B provide data demonstrating that functionalized fullerene modulates RF heating rate. FIG. 8A provides a structure of C₆₀-ser. FIG. 8B shows that C₆₀-ser (∘), shown in comparison to propylene glycol (□), enhances the heating rate of PBS (▪) by reducing its conductivity. All heating rates reported are averages of three replicates with error bars indicating SD. The error bars may be smaller than symbols.

FIG. 9 shows a generalized aqueous RF heating curve and its application. Peak aqueous RF heating occurs at 0.06 S/m. Addition of salts, kosmotropic materials, or C₆₀-ser results in shift of conductivity and heating rate, as indicated by arrows. The location of materials of interest on the curve are shown, including the range occupied by nanoparticles (NPs=AuNPs, SWNTs) and the conductivity range of many human tissues.

FIGS. 10A-D show data relating to the complex permittivity of NaCl. Shown are data relating to real-valued permittivity (FIG. 10A), imaginary permittivity (FIG. 10B), and conductivity (FIG. 10C) of 0.1 (black), 1 (red), 10 (blue), and 100 (grey) mM NaCl over the frequency range of 10 MHz to 3 GHz. Dashed blue and grey lines indicate low frequency polarization correction of real-valued permittivity of 10 and 100 mM NaCl, respectively. Vertical line indicates RF operating frequency of 13.56 MHz. FIG. 10D shows concentration dependence of real permittivity ∈′ᵣ (circles) and imaginary permittivity ∈″ᵣ (squares) for NaCl at 13.56 MHz. Both measured (black) and corrected (red) values are shown. The absolute value of the relative permittivity |∈ᵣ| is indicated for measured (grey line) and corrected (dotted red line) values. Differences between corrected and uncorrected values of ∈″ᵣ and |∈ᵣ| are less than 1.2%. The sudden increase in |∈ᵣ| around 5 mM NaCl is responsible for the sharp decrease in E_{eff}, which causes peak RF heating behavior.

FIGS. 11A-C show the RF heating rates of salt solutions as a function of concentration and imaginary permittivity. FIG. 11A shows the heating rates of trivalent (blue circles), divalent (red triangles) and monovalent (black squares) salts as a function of concentration. Solid lines indicate Gaussian fit for that salt valency. FIG. 11B shows the empirical heating rates for all salts as a function of DC conductivity. FIG. 11C shows empirical heating rates for all salts as a function of imaginary permittivity. In all plots, solid lines indicates Gaussian fit and symbols indicate the average heating rate from three experiments. Error bars are omitted for clarity, but are typically smaller than the symbols. Dashed lines indicate peak heating permittivity or conductivity.

FIGS. 12A-B show the RF heating rates of salt solutions as a function of concentration (FIG. 12A) and conductivity (FIG. 12B) at 100 W. In FIG. 12B, materials of interest such as water, blood, normal saline, and phosphate buffered saline (PBS) are indicated on the heating curve along with heating rate model fit (red line).

FIGS. 13A-B compare a fitting heating rate model to heating rates of salt solutions. Heating rates of salt solutions (black circles) and fitted heating rate model (red line) as a function of imaginary permittivity at 100 W (FIG. 13A) and 900 W (FIG. 13B) are shown. At 900 W, heating rates are calculated from the first five seconds of RF exposure.

FIGS. 14A-B provide data relating to the adjustment of the dielectric properties of aqueous solutions to enhance heating rate. FIG. 14A provides data of conductivity-dependent RF heating rates at 100 W of numerous additives at 100, 250, and 500 mg/mL in PBS. FIG. 14B provides comparative data of conductivity-dependent RF heating rates at 100 W of C₆₀-serinol (C₆₀-ser) and propylene glycol at 25, 50, and 100 mg/mL.

FIGS. 15A-C provide data relating to methods of adjusting the dielectric properties of blood to optimize RF heating rates. Differences in conductivity (FIG. 15A) and heating rate (FIG. 15B) of blood upon addition by weight % of water (blue), normal saline (red), glycerol (orange), and propylene glycol (grey) are shown. FIG. 15C shows the conductivity dependent heating rates of blood with different additives at 5%, 10%, 15%, and 20% additive content. Arrows indicate increase in additive content. All reported measurements are averages of three replicates with error bars indicating standard deviation. Error bars in C are omitted for clarity but are generally smaller than the symbols.

FIG. 16 shows an RF heating rate with respect to both conductivity and real permittivity. Shown are theoretical RF heating rates at 100 W, assuming constant density and specific heat capacity (equal to those of water) and an applied electric field of 490 kV/m.

FIGS. 17A-C show an RF heating rate of materials with low real permittivity. FIG. 17A shows the frequency-dependent ∈′ of mixtures of water, ethanol (EtOH) and propylene glycol (PG). As water content decreases, so does ∈′. Also shown are RF heating rates at 100 W as a function of ∈″ of water/PG mixtures (FIG. 17B) and water/EtOH mixtures (FIG. 17C) containing different salt concentrations. Percentages indicate PG or EtOH content of the bulk mixture. The increasing ∈″ corresponds to greater salt concentration. Solid lines are the heating rate model fit for the corresponding curve.

FIGS. 18A-C show dielectric and RF heating properties of oils. Frequency-dependent real (FIG. 18A) and imaginary (FIG. 18B) permittivity of ethiodol and vegetable oil are shown. FIG. 18C shows a comparison of ethiodol RF heating rate at 900 W with aqueous salt solutions as a function of imaginary permittivity.

FIG. 19 shows the increase of real permittivity (∈′) of 50 mM NaCl upon addition of the following methylated glycine derivatives: glycine, sarcosine (N-methylglycine), N,N-dimethylglycine, and glycine betaine (N,N,N-trimethylglycine).

FIGS. 20A-D show characterization data of Pluronic-wrapped metallic and semiconducting single-walled carbon nanotubes (m-/s-SWNTs), as acquired using Raman spectroscopy (FIG. 20A), UV-VIS (FIG. 20B), atomic force microscopy (AFM) (m-SWNTs) (FIG. 20C), and scanning electron microscopy (SEM) (s-SWNTs) (FIG. 20D).

FIGS. 21A-B show m-/s-SWNT heating data. FIG. 21A shows derived heating rates (° C./s) versus Pluronic wrapped (2% w/v) m-/s-SWNTs at concentrations of 1, 10, 25, 50, and 100 mg/L. FIG. 21B provides an example of raw data used to calculate the heating rate in FIG. 21A for a 100 mg/L SWNT concentration. SWNT solutions (red=semiconducting, green=metallic) were exposed to the RF field. The SWNTs were then removed and the supernatant (SN) re-exposed. Also shown for reference is a non-SWNT Pluronic F108 solution (grey image, blue data line) at the same polymer concentration (2% w/v).

FIGS. 22A-B show data relating to m-/s-SWNT alignment. FIG. 22A shows optical and IR data of m-/s-SWNTs heating up and aligning in an RF field as a function of time. Direction of the RF electric-field vector is also shown in the top right-hand corner. FIG. 22B shows that the alignment was modulated by turning on-and-off the RF field. The SWNTs fall back into suspension when the RF field is off and align as soon as the RF field is turned back on. The angle between IR and optical camera images was ˜30°. Blue/Green/and Red areas cover the temperature range of 21° C. to 50° C.

FIG. 23 shows the heating rates of gold nanoparticles (AuNPs) and m-/s-SWNT as a function of total surface area in a 1.3 ml aqueous sample. Linear regression R² values are also provided.

FIGS. 24A-B show the heating rate of various suspensions. FIG. 24A shows the heating rates of Pluronic wrapped m-/s-SWNT suspensions (100 mg/L) versus NaCl molarity (includes NaCl control and filtered supernatants, SN). The grey shaded area depicts the region of biological relevance where the NaCl molarity mimics the biological host conductivity (σbio) range of 0.01 to 5.0 S/m. FIG. 24B shows the heating rate difference between SWNT suspensions and filtered supernatants.

FIGS. 25A-C provide comparative data and images of various SWNT solutions. FIG. 25A shows that the heating rates of saline solutions (154 mM NaCl) show no enhancement with addition of high concentrations of CNI-SWNTs (1100 mg/L). Pluronic F108 concentrations (w/v) of 0.02% and 2% also have no effect on heating. FIG. 25B shows that CNI-SWNTs suspended in 0.02% Pluronic precipitate out of solution after 3 minutes. FIG. 25C shows that 2% F108 allows for homogenous CNI-SWNTs dispersions, even after 24 hours.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose.

Aqueous systems, regardless of composition, heat according to bulk dielectric properties under radiofrequency (RF) exposure at 13.56 MHz. Moreover, peak heating for aqueous systems occurs at 0.06 S/m, beyond which increases in conductivity result in decreases in heating rate. Biologically relevant systems, such as blood and body fluids, exceed the aforementioned peak heating conductivity, precluding the use of conductive materials for heating rate enhancement.

For instance, hyperthermia has been used for cancer treatment because cancer cells are more hyperthermic to heat than healthy cells. However, most tumors cannot be treated by hyperthermia non-invasively. For this reason, the Kanzius external RF generator, which takes advantage of the tissue penetrating properties of RF energy, has been proposed for inducing hyperthermia in tumors in a safe and non-invasive fashion.

Studies in mice have shown that the Kanzius system selectively enhances hyperthermia in cancerous tissue over healthy tissue. In addition, numerous studies have coupled RF exposure with nanoparticles, such as gold nanoparticles and single-walled carbon nanotubes. However, the use of such nanoparticles in enhancing treatment efficacies of biological systems has not been effectively demonstrated. As such, a need exists for more improved methods of optimizing the treatment efficacy of various biological systems. The present disclosure addresses this need.

In some embodiments, the present disclosure pertains to methods of optimizing a treatment efficacy of a biological system. In some embodiments, the methods of the present disclosure include tuning a property of the biological system by adding an optimizing agent to the biological system. In some embodiments, the tuned biological system is then treated in various manners.

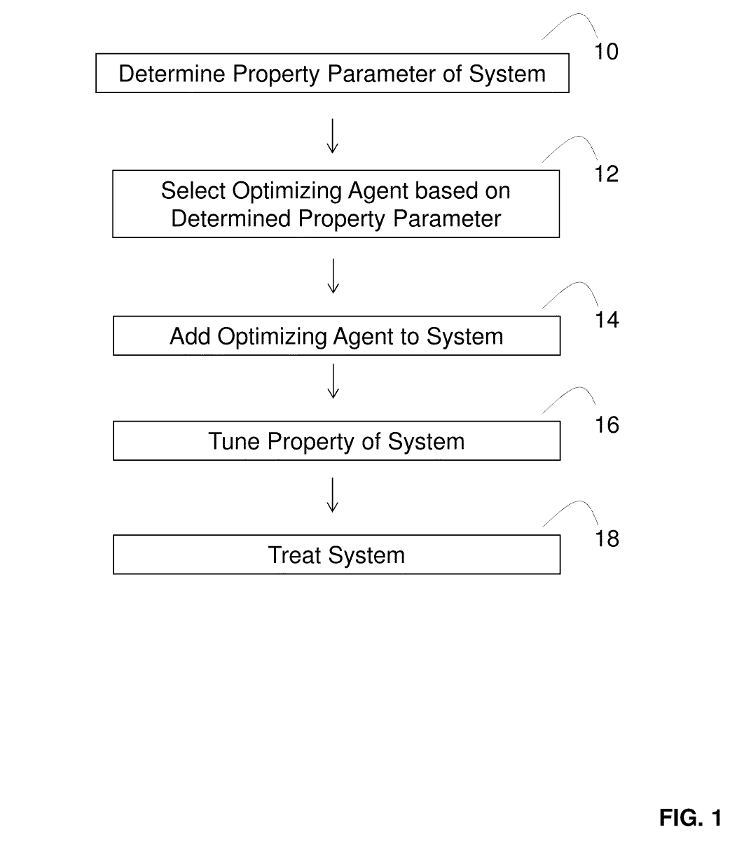

In some embodiments illustrated in FIG. 1, the methods of the present disclosure include steps of determining a property parameter of a biological system (step 10); selecting an optimizing agent based on the determined property parameter (step 12); and adding the optimizing agent to the biological system (step 14), thereby tuning a property of the biological system (step 16). In some embodiments, the methods of the present disclosure also include a step of treating the biological system (step 18).

As set forth in more detail herein, the methods of the present disclosure can be used to add various optimizing agents to various biological systems. Moreover, the methods of the present disclosure may be utilized to optimize various treatment efficacies by tuning various properties of the biological systems.

Biological Systems

Biological systems generally refer to systems that directly or indirectly relate to living organisms. The methods of the present disclosure can be utilized to optimize the treatment efficacies of various biological systems. For instance, in some embodiments, the biological system includes, without limitation, aqueous biological systems, body fluids, blood, solid biological systems, tissues, organs, vasculatures, matrices, and combinations thereof. In some embodiments, the biological system includes matrices, such as intracellular matrices, extracellular matrices, and combinations thereof. In some embodiments, the biological system includes vasculatures, such as local tumor vasculatures.

In some embodiments, the biological system includes a tissue. In some embodiments, the tissue includes, without limitation, human tissues, animal tissues, tumors, tumor-containing tissues, natural tissues, synthetic tissues, fatty tissues, non-fatty tissues, and combinations thereof.

In some embodiments, the biological system is part of a subject or isolated from the subject. In some embodiments, the subject is a human being suffering from a condition. In some embodiments, the condition includes, without limitation, cancer, tumor growth, infectious diseases, inflammatory conditions, and combinations thereof. In some embodiments, the condition is cancer.

Addition of Optimizing Agents to Biological Systems

Various methods may be utilized to add optimizing agents to biological systems. For instance, in some embodiments, the addition includes contacting the biological system with the optimizing agent. In some embodiments, the contacting occurs by the direct application of the optimizing agent to the biological system. In some embodiments, the contacting includes the application of the optimizing agent to a subject containing the biological system. In some embodiments, the application of the optimizing agent to the subject includes, without limitation, oral administration, inhalation, subcutaneous administration, intravenous administration, intraperitoneal administration, intramuscular administration, intrathecal injection, topical administration, and combinations thereof.

Optimizing Agents

Optimizing agents generally refer to compounds that are capable of tuning a property of a biological system. The methods of the present disclosure may utilize various types of optimizing agents. In some embodiments, the optimizing agents of the present disclosure are inexpensive and widely available. In some embodiments, the optimizing agents of the present disclosure exclude expensive or complex drugs. In some embodiments, the optimizing agents of the present disclosure are biocompatible, non-toxic, and readily metabolized. In some embodiments, the optimizing agents of the present disclosure include, without limitation, kosmotropic materials, non-polar materials, carbon nanotubes, salts, and combinations thereof.

In some embodiments, the optimizing agents of the present disclosure include a kosmotropic material. Kosmotropic materials generally refer to compounds that stabilize a water network that is associated with a biological system. In some embodiments, kosmotropic materials stabilize a water network by preferentially interacting with water molecules and forming hydrogen bonds to water that are stronger than the water-water hydrogen bonds.

The optimizing agents of the present disclosure can include various kosmotropic materials. For instance, in some embodiments, the kosmotropic materials include, without limitation, alcohols, zwitterionic molecules, amines, neutral molecules, water soluble molecules, water soluble nanomaterials, carbohydrates, amino acids, organosulfur compounds, and combinations thereof.

In some embodiments, the kosmotropic materials include an organosulfur compound. In some embodiments, the organosulfur compound is dimethyl sulfoxide.

In some embodiments, the kosmotropic material includes a carbohydrate. In some embodiments, the carbohydrate includes, without limitation, maltose, glucose, sucrose, trehalose, and combinations thereof.

In some embodiments, the kosmotropic material includes an alcohol. In some embodiments, the alcohol includes, without limitation, methanol, glycerol, polyhydric alcohols, sorbitol, glycols, ethylene glycol, propylene glycol, and combinations thereof.

In some embodiments, the kosmotropic material includes a water soluble nanomaterial. In some embodiments, the water soluble nanomaterial includes, without limitation, functionalized fullerenes, functionalized carbon nanotubes, shortened carbon nanotubes, derivatized nanoparticles, and combinations thereof.

In some embodiments, the kosmotropic material includes a nanoparticle, such as a derivatized nanoparticle. In some embodiments, nanoparticles may be derivatized with one or more functionalizing agents in order to optimize their properties. In some embodiments, the nanoparticles may be derivatized with functionalizing agents through covalent or non-covalent bonds. In some embodiments, the derivatized nanoparticle includes, without limitation, gold nanoparticles, silicon oxide (SiOₓ) nanoparticles, tannic acid-functionalized nanoparticles, citrate-functionalized nanoparticles, magnetic nanoparticles, and combinations thereof.

In some embodiments, nanoparticles may also be loaded with one or more optimizing agents (e.g., kosmotropes, fats, lipids, and carbohydrates) in order to optimize their properties. In some embodiments, the loading of nanoparticles with optimizing agents can occur by covalent or non-covalent loadings.

In some embodiments, the kosmotropic material includes an amine. In some embodiments, the amine includes, without limitation, zwitterionic amines, betaines, glycine, methylated glycine, proline, sarcosine, N,N-dimethyl glycine, betaines, glycine betaine, proline betaine, trimethylamine oxide, and combinations thereof.

In some embodiments, the optimizing agent includes carbon nanotubes. In some embodiments, the carbon nanotubes include, without limitation, single-walled carbon nanotubes, multi-walled carbon nanotubes, metallic carbon nanotubes, semiconducting carbon nanotubes, and combinations thereof.

In some embodiments, the optimizing agents of the present disclosure include metallic or semiconducting single-walled carbon nanotubes. In some embodiments, the optimizing agents of the present disclosure include Pluronic wrapped carbon nanotubes.

In some embodiments, the optimizing agent includes a non-polar material. In some embodiments, the non-polar material includes, without limitation, lipids, oils, fats, porphyrins, aromatic compounds, ethiodol (Lipiodol), and combinations thereof.

In some embodiments, the optimizing agents of the present disclosure include a salt (i.e., a physiologically acceptable salt). Various salts may be used as optimizing agents. For instance, in some embodiments, the salt includes, without limitation, inorganic salts, metallic salt solutions, non-metallic salt solutions, electrolyte solutions, monovalent salts, divalent salts, trivalent salts, and combinations thereof.

In some embodiments, the salt includes monovalent salts. In some embodiments, the monovalent salts include, without limitation, LiCl, NaCl, KCl, NaBr, NaI, NaOAc, NaNO₃, NaC₂H₃O₂, CsCl, and combinations thereof.

In some embodiments, the salt includes multi-valent salts. In some embodiments, the salt includes divalent salts. In some embodiments, the divalent salts include, without limitation, MgCl₂, CaCl₂, BaCl₂, Na₂SO₄, Na₂CO₃, and combinations thereof.

In some embodiments, the salt includes trivalent salts. In some embodiments, the trivalent salts include, without limitation, FeCl₃, AuCl₃, GdCl₃, Na₃(C₆H₅O₇), Na₃PO₄, and combinations thereof.

The optimizing agents of the present disclosure can be added to biological systems at various concentrations. For instance, in some embodiments, optimizing agents are added to a biological system at concentrations that range from about 1 mg/mL to about 1,000 mg/mL. In some embodiments, optimizing agents are added to a biological system at concentrations that range from about 25 mg/mL to about 500 mg/mL. In some embodiments, optimizing agents are added to a biological system at concentrations that range from about 1 mg/mL to about 100 mg/mL. In some embodiments, optimizing agents are added to a biological system at concentrations that range from about 1 mg/mL to about 50 mg/mL. In some embodiments, optimizing agents are added to a biological system at concentrations that range from about 50 mg/mL to about 250 mg/mL. In some embodiments, optimizing agents are added to a biological system at concentrations that range from about 100 mg/mL to about 200 mg/mL.

In some embodiments, optimizing agents are added to a biological system at concentrations that range from about 1 nM to about 100 mM. In some embodiments, optimizing agents are added to a biological system at concentrations that range from about 5 nM to about 50 nM. In some embodiments, optimizing agents are added to a biological system at concentrations that range from about 10 nM to about 20 nM. In some embodiments, optimizing agents are added to a biological system at concentrations that range from about 1 mM to about 100 mM. In some embodiments, optimizing agents are added to a biological system at concentrations that range from about 1 mM to about 10 mM.

In some embodiments, optimizing agents are added to a biological system at concentrations that range from about 1 wt % to about 50 wt %. In some embodiments, optimizing agents are added to a biological system at concentrations that range from about 1 wt % to about 25 wt %. In some embodiments, optimizing agents are added to a biological system at concentrations that range from about 1 wt % to about 20 wt %. In some embodiments, optimizing agents are added to a biological system at concentrations that range from about 1 wt % to about 15 wt %. In some embodiments, optimizing agents are added to a biological system at concentrations that range from about 5 wt % to about 15 wt %.

Tuned Properties of Biological Systems

The optimizing agents of the present disclosure can be utilized to tune various properties of a biological system. As used herein, “tune”, “tuning” and like terms mean to modulate a selected property of a biological system to enhance the efficacy of a treatment carried out on a subject in need thereof. For instance, in some embodiments, the tuned property includes, without limitation, dielectric properties of the biological system, physical properties of the biological system, the heating rate of the biological system, the heat capacity of the biological system, the conductivity of the biological system, the permittivity of the biological system, the density of the biological system, the ionic mobility through the biological system, and combinations thereof.

In some embodiments, the tuned property includes the heating rate of the biological system. In some embodiments, the tuned property includes the heating rate of the biological system exposed to a radiofrequency (RF) field.

The tuning of a property of a biological system can have various effects on the biological system. For instance, in some embodiments, the tuning decreases the permittivity of the biological system. In some embodiments, the tuning reduces the ionic mobility through the biological system.

In some embodiments, the tuning decreases the heating rate of a biological system. In some embodiments, the tuning increases the heating rate of the biological system. In some embodiments, the tuning increases the heating rate of the biological system by about 10% to about 80%. In some embodiments, the tuning increases the heating rate of the biological system by at least about 50%. In some embodiments, the tuning increases the heating rate of the biological system by at least about 60%. In some embodiments, the tuning increases the heating rate of the biological system by at least about 62%.

In some embodiments, the tuning increases the conductivity of a biological system. In some embodiments, the tuning reduces the conductivity of a biological system. For instance, in some embodiments, the tuning reduces the conductivity of the biological system by about 50% to about 300%. In some embodiments, the tuning reduces the conductivity of the biological system by at least about 100%. In some embodiments, the tuning reduces the conductivity of the biological system by at least about 200%. In some embodiments, the tuning reduces the conductivity of the biological system by about 233%. In some embodiments, the reduced conductivity enhances the heating rate of the biological system.

Tuning Methods

Various methods may be utilized to tune the properties of biological systems. For instance, in some embodiments, the tuning occurs by: (a) determining a property parameter of the biological system; (b) selecting the optimizing agent to be added to the biological system based on the determined property parameter; and (c) adding the optimizing agent to the biological system.

In some embodiments, a property parameter of a biological system is determined by measuring the property parameter, such as through the use of magnetic resonance imaging (MRI) or other testing methods. In some embodiments, a property parameter of a biological system is determined by estimating the property parameter. In some embodiments, the estimation occurs by approximation from known values. In some embodiments, the estimation occurs by calculating the heating rate of a biological system exposed to a radiofrequency (RF) field from its dielectric and physical properties.

In some embodiments, the property of a biological system is tuned in a selective manner. For instance, in some embodiments, the property of a first biological system is tuned while the property of a second biological system remains substantially the same. In some embodiments, the first biological system is associated with a condition while the second biological system is not associated with the condition. In some embodiments, the selective tuning does not optimize the property of the second biological system. In some embodiments, the first biological system is a tumor while the second biological system is normal tissue (e.g., normal tissue nearby the tumor).

In some embodiments, the selective tuning occurs due to a better absorption of the optimizing agent by the first biological system. For instance, in some embodiments, a tumor (i.e., a first biological system) can absorb optimizing agents more effectively than normal tissue (i.e., a second biological system). In some embodiments, the selective tuning occurs due to the different effects of the optimizing agent on the first and second biological systems. For instance, in some embodiments, an optimizing agent may make a tumor (i.e., a first biological system) more hyperthermic than normal tissue (i.e., a second biological system).

Treatment Methods

The methods of the present disclosure can be utilized to optimize the efficacy of various biological system treatments. In additional embodiments, the methods of the present disclosure also include a step of treating the biological system.

In some embodiments, the optimized treatment includes radiation treatment of the biological system. In some embodiments, the optimized treatment includes heat treatment of the biological system. In some embodiments, the optimized heat treatment includes hyperthermia treatment of the biological system. In some embodiments, the optimized treatment occurs in a radiofrequency field. In some embodiments, the optimized treatment includes a radiofrequency-induced hyperthermia treatment of a biological system. In some embodiments, the optimized treatment takes advantage of dilated tumor blood vessel networks by radiofrequency field treatment for enhanced delivery of drugs and optimizing agents. Additional optimized treatments can also be envisioned.

Applications and Advantages

The methods of the present disclosure can have various advantages. For instance, in some embodiments, the optimized treatment methods of the present disclosure can enhance tumor reduction by various mechanisms, such as by increasing the tumor heating rate. In some embodiments, the methods of the present disclosure can be utilized to optimize treatment efficacies of targeted biological systems (e.g., cancerous tissues) while minimizing damage to untargeted biological systems (e.g., healthy tissues). In some embodiments, the methods of the present disclosure can be utilized to enhance the heating rate of cancerous tissues while minimizing the heating rates of healthy tissues.

Moreover, the methods of the present disclosure can occur without the need for expensive or complex drugs. Rather, in some embodiments, the optimizing agents of the present disclosure can be widely available, inexpensive, and generally safe. Furthermore, side effects of the methods of the present disclosure can be minimal or non-existent.

Without being bound by theory, Applicants envision that kosmotropic materials confer many of the aforementioned advantageous properties. For instance, when added to a biological system, kosmotropic materials can decrease the biological system's conductivity, permittivity, and ionic mobility. This in turn can increase the radiofrequency heating rate of the biological system.

As such, the methods of the present disclosure can have various applications. For instance, in some embodiments, the methods of the present disclosure can be utilized to optimize tissue heating rates during noninvasive radiofrequency (RF)-induced hyperthermia treatment of cancer. In some embodiments, the methods of the present can provide low cost and readily available treatment options for a range of cancers or other diseases without the need for expensive drugs. Moreover, the methods of the present disclosure can be made available to developing regions of the world in a facile and affordable manner.

Reference will now be made to more specific embodiments of the present disclosure and experimental results that provide support for such embodiments. However, Applicants note that the disclosure below is for illustrative purposes only and is not intended to limit the scope of the claimed subject matter in any way.

### Example 1

### Optimizing Noninvasive Radiofrequency Cancer Hyperthermia

In this Example, Applicants demonstrate that kosmotropic materials, including sugars, glycols, zwitterionic molecules, and a water-soluble fullerene, can be added to aqueous systems to reduce conductivity and enhance radiofrequency heating rate.

In particular, Applicants show in this Example that kosmotropic materials can be used to reduce the conductivity of biological systems and enhance their bulk heating rate accordingly. Applicants also identify a water-soluble fullerene as the first nanomaterial used for bulk RF heating enhancement of a highly conductive aqueous system.

Experimental RF heating curves for a number of salt solutions are presented in this Example. The results show that heating corresponds to the dielectric loss mechanism, with peak heating occurring at a conductivity of 0.06 S/m at 13.56 MHz, regardless of the identity of the salt. Applicants also show that this heating mechanism applies to complex aqueous systems, such as buffers and blood, and even to nanoparticle suspensions. Such nanoparticles do not contribute to bulk heating except by their contribution to the bulk dielectric properties of water, which in turn determine the heating of the material according to simple dielectric heating.

### Example 1.1

### RF Heating by Dielectric Loss Mechanism

Macroscopic materials respond to electromagnetic (EM) fields as governed by their dielectric properties (i.e., permittivity and conductivity). In time-varying alternating electric fields such as radiofrequency waves, a material's permittivity becomes a complex function as a result of the electric polarization of the medium and is given by Equation 1:

$${ɛ}_{r} = {ɛ}_{r}^{′} + i {ɛ}_{r}^{″} = {ɛ}_{r}^{′} + i \frac{σ}{{ωɛ}_{0}} \qquad ( 1 )$$

In Equation 1, ∈ᵣ, ∈′ᵣ, ∈″ᵣ are the complex, real-valued, and imaginary relative permittivities, respectively. In addition, i is \sqrt{−1}, σ is the conductivity, ∈₀ is the relative permittivity of free space, and co is the angular frequency. The energy absorbed by a dielectric material exposed to EM fields within the radio- and microwave frequency range (3 kHz-300 GHz) is defined by the specific absorption rate (SAR, where SAR=σE²/2ρ). The rate of heating of a material exposed to EM fields is characterized by Equation 2:

$$HR = \frac{\partial T}{\partial t} = \frac{σ {\vert {E}_{eff} \vert}^{2}}{2 ρ {c}_{p}} \qquad ( 2 )$$

In Equation 2, E_{eff} is the effective electric field within the sample, ρ is the mass density, and cₚ is the specific heat capacity. The effective electric field can vary greatly across different materials under exposure to EM fields. When this variance is not adequately accounted for, a potential mistake in interpreting equation (2) is to equate higher conductivity with increased heating rate. However, the permittivity mismatch between the sample and the surrounding medium, such as saline and air, greatly reduces the effective electric field within the sample. Ultimately, the effective electric field can vary greatly and is dependent on the applied electric field strength, the dielectric properties of the material, and even the size and shape of the material.

As illustrated in Equation 3, E_{eff} is related to the applied electric field (Eₐₚₚ):

$${E}_{eff} = \frac{1}{1 + N ( \frac{{ɛ}_{r}}{{ɛ}_{r , air}} - 1 )} {E}_{app} \qquad ( 3 )$$

In Equation 3, N is the dimensionless polarization tensor that accounts for the geometry of the sample. Likewise, ∈ᵣ and ∈_{r,air} represent the relative permittivity of the material of interest and surrounding medium, respectively.

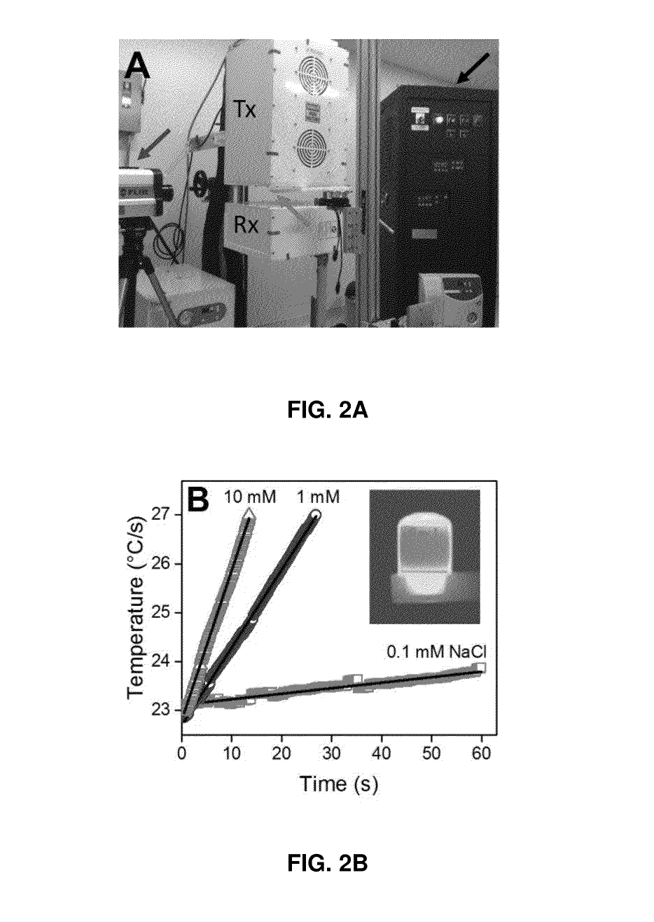

In the case presented in FIGS. 2A-B of a cylindrical cuvette of diameter 10 mm and height 18 mm, N is estimated to be 0.1986. In particular, FIG. 2A shows the RF generator used to create a high-voltage electric field (Eₐₚₚ) in the 10 cm air gap between the transmitting (Tx) and receiving (Rx). The sample, loaded onto a 1.3 mL non-conducting quartz cuvette placed approximately 8 mm from the transmitting head, experiences a reduced effective electric field (E_{eff}) due to the large difference in permittivity between the sample and surrounding air.

### Example 1.2

### RF Heating of Aqueous Systems

Applicants measured the RF heating rates of an assortment of salts dissolved in ultra-pure water in the concentration range of about 0.01 mM to about 200 mM. The salts tested were NaCl, NaC₂H₃O₂, MgCl₂, Na₂SO₄, AuCl₃, and Na₃C₆H₅O₇ (sodium citrate). Samples were exposed to a RF field (13.56 MHz operating frequency) at 100 W generator power (FIG. 2A).

Sample temperature was measured using an infrared camera. The heating rates were calculated by a least-squares linear regression of the resultant temperature-time plot (FIG. 2B). Samples were heated from 23° C. to either 27° C. or for a duration of 60 seconds, whichever occurred first. Over this selected small temperature range, the variation of the temperature-dependence of dielectric properties was negligible.

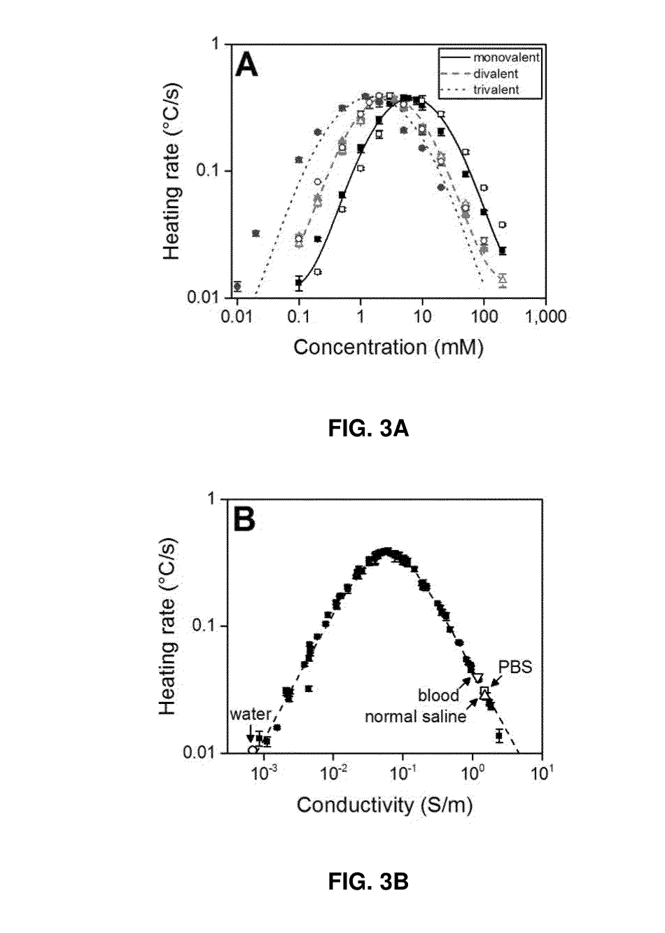

Moreover, each salt displayed a characteristic peak heating curve as a function of concentration (FIG. 3A). In addition, salts of greater valence typically displayed peak heating at lower concentrations than salts of lesser valence.

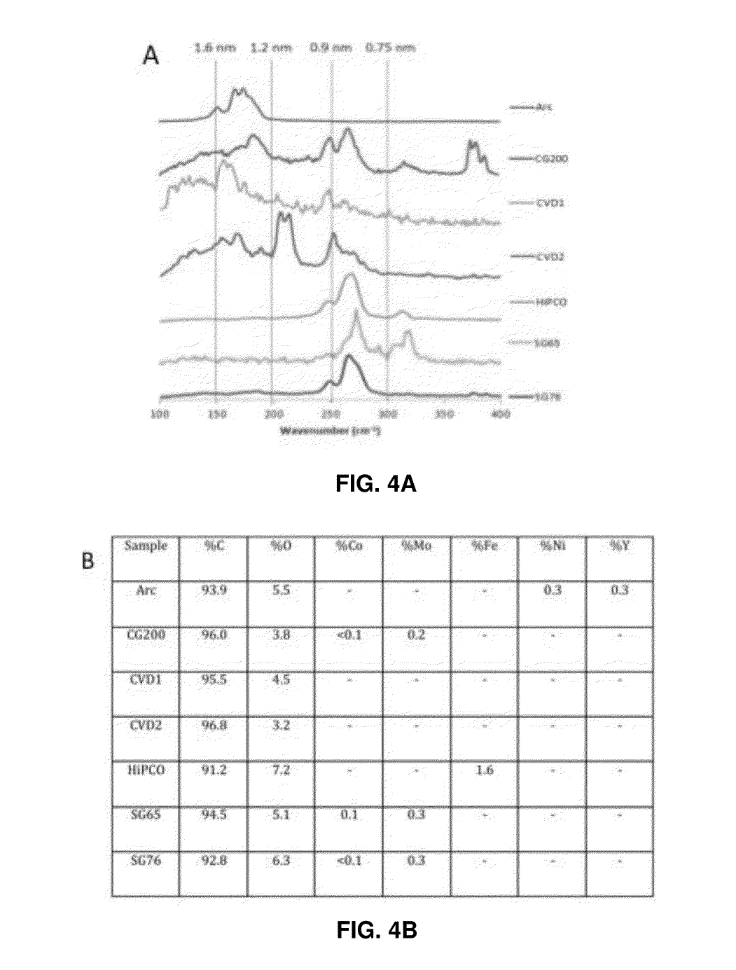

The complex relative permittivity (∈′ᵣ and e″ᵣ) of all samples was measured over the frequency range of 10 MHz to 3 GHz (FIGS. 4A-B). From these measurements, the conductivity was obtained using the relationship σ=ω∈_{D}∈″ᵣ. Given the RF operating frequency of 13.56 MHz, only the conductivity values for this frequency were considered throughout this Example.

The unique heating curves for each individual salt collapse into a single heating curve when the conductivity of solutions is considered (FIG. 3B). Peak heating of ˜0.39° C./s occurred at a conductivity of 0.060 S/m for all salts studied.

The aforementioned results indicate that the saline heating model can include many aqueous solutions by demonstrating that heating rate is dependent only on bulk conductivity, regardless of specific atomic or molecular identity. More complicated systems, such as buffer solutions and blood, also heat according to this heating curve, demonstrating the application of this model to many aqueous systems, regardless of their content (FIG. 3B). As described in more detail herein, the aforementioned model also applies to nanoparticle suspensions, including gold nanoparticles (AuNPs) and single-walled carbon nanotubes (SWNTs), which have a long history of debate regarding RF heating mechanisms.

Difficulties exist in measuring the applied electric field (Eₐₚₚ) when the field strength is strong. Attempts to measure the field with a voltage probe have led to rapid (<1 second) overheating and subsequent damage of the probe, even at RF generator power below 10 W. Instead, Applicants fit a heating rate model described by equations (2) and (3) to the salt heating data (HR, σ and ∈ᵣ) using Eₐₚₚ as the fit parameter. By this method, Applicants found a field strength of 163 kV/m.

The same model can be applied to the low power 13.56 MHz RF system used previously (J. Appl. Phys. 113, 074902 (2013)). This system generated a maximum temperature increase in saline of only 0.7° C. over an RF exposure period of 10 minutes (0.0012° C./s). Based on the sample dimensions provided (diameter 35 mm and height 4 mm), Applicants estimate N for the prior system to be 0.7416 and calculate Eₐₚₚ to be 33.5 kV/m, in agreement with the electric field strength estimated by the authors from low power measurements.

### Example 1.3

### Optimizing RF Heating Behavior

From the generalized RF heating curve for salts, it is apparent that an increase in material conductivity does not monotonically increase heating rate. Peak heating for aqueous media occurs at a conductivity of 0.060 S/m, beyond which addition of conductive material will decrease heating rate. Indeed, this was shown using (s/m-SWNTs), though this behavior was not yet well understood. Blood and body fluids are present on the far right of the curve with conductivities of 1.1 and 1.5 S/m, respectively, at 13.56 MHz. Intra- and extra-cellular environments of cells have similarly high ion content. Therefore, increasing the heating rate of body tissues under RF energy requires diluting existing ionic content, a task not readily performable nor tolerated in vivo.

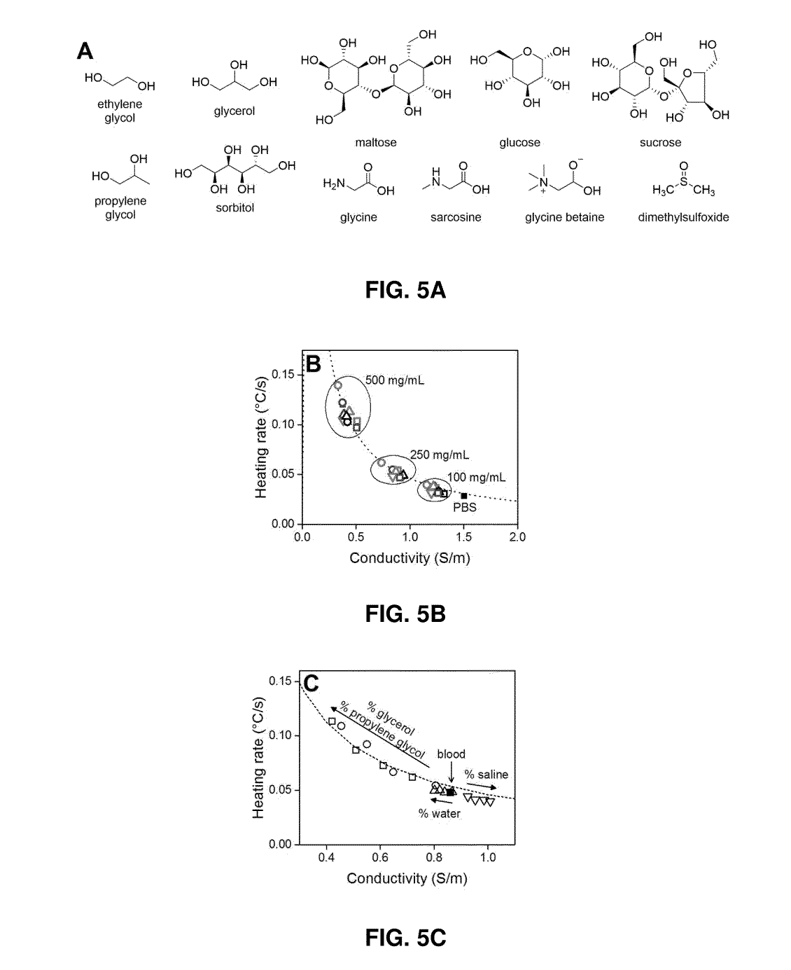

An alternative approach presented in this Example is to introduce kosmotropic materials. A number of kosmotropic materials used in this study are shown in FIG. 5A. To Applicants' knowledge, this is the first time that the RF heating rates of aqueous solutions have been modulated by kosmotropic materials.

Each of the materials listed in FIG. 5A were dissolved at 100, 250, and 500 mg/mL in phosphate buffered saline (PBS), a biologically iso-osmolar solution with similar conductivity (1.15 S/m) to blood and body tissues. To maintain equivalent ionic concentrations in all samples, these solutions were prepared and appropriately diluted from concentrated stock PBS (10×PBS containing 90 g/L NaCl, 1.44 g/L KH₂PO₄, and 7.95 g/L Na₂HPO₄). RF heating rates and conductivity were measured according to the procedure described in Examples 1.1 and 1.2. Addition of kosmotropic materials increased the heating rate of PBS in a concentration dependent manner as a result of a corresponding decrease in conductivity (FIG. 5B).

Propylene glycol outperformed all other materials at every concentration studied, increasing the heating rate of PBS by 490% when added at a concentration of 500 mg/m. On average, PBS heating rate increased from 0.028° C./s to 0.034±0.001, 0.052±0.001, and 0.112±0.004° C./s at kosmotropic material concentrations of 100, 250, and 500 mg/mL, respectively. All of the solutions tested can be found on the same heating curve as simple saline solutions, again demonstrating the universality of the aqueous heating model.

In a similar study, various additives were added to bovine blood containing 2-3% ethylenediamine tetraacetic acid (EDTA) to prevent coagulation. The additives tested were propylene glycol, glycerol, pure water and normal saline at concentrations of 1-20% by weight. Addition of propylene glycol and glycerol resulted in a decrease in blood conductivity and increase in heating rate (FIG. 5C).

A significant increase in heating rate (p<0.005) was observed starting at 5% propylene glycol and 10% glycerol. In contrast, addition of normal saline (1.49 S/m, 0.031° C./s) resulted in opposite trends in conductivity and heating, although the changes were not significant. Addition of pure water had similar, though reduced, effects to those of propylene glycol and glycerol on blood. Although these three additives all dilute the ionic content of blood and are nonconductive, only propylene glycol and glycerol significantly modulate conductivity and heating. Without being bound by theory, it is envisioned that this difference in conductivity attenuation is attributed to the water-structuring properties of these materials.

FIG. 5C demonstrates that it is possible to modulate the heating rate of a material by using different additives. In this case, the initial or unaltered conductivity and heating rate of blood are 0.86 S/m and 0.045° C./s, respectively. Addition of glycerol and propylene glycol shifts blood to the left of the curve towards greater heating rates. The same effect is true for water, although to a much lesser degree. However, the opposite effect is observed for saline, which shifts blood to the right of the curve towards reduced heating rates.

### Example 1.4

### RF Heating of Nanoparticle Suspensions

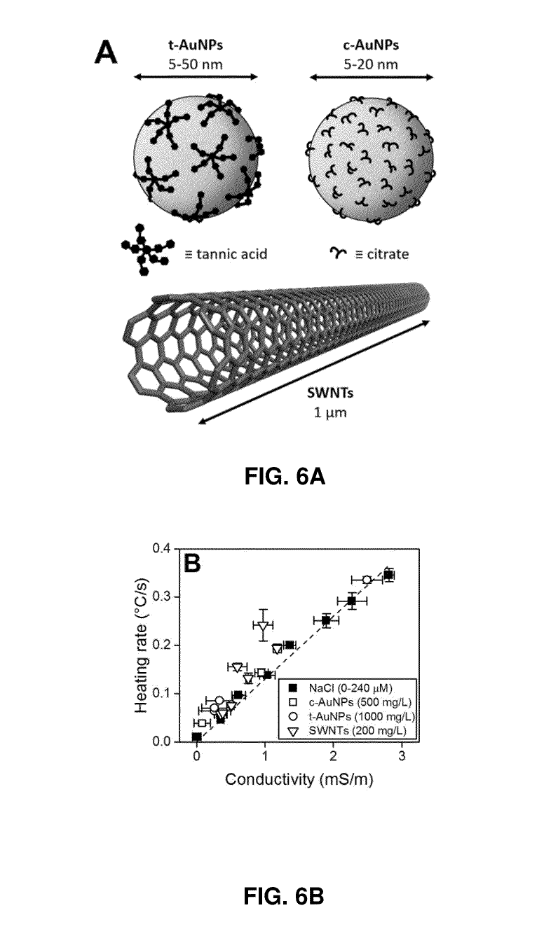

Next, Applicants used the RF system and dielectric characterization described previously to investigate the RF-induced heating rates of a number of nanoparticle suspensions (FIG. 6A). Spherical citrate-capped gold nanoparticles (c-AuNPs) were obtained in 5 and 20 nm diameter. Tannic acid-capped gold nanoparticles (t-AuNPs) were obtained in 5, 10, 20, and 50 nm diameter. Full length single-walled carbon nanotubes (SWNTs) were obtained with an average length of 1 μm. Following purification, all nanoparticles were re-suspended in ultra-pure water for dielectric and RF heating analysis. Nanoparticle concentrations were as follows: c-AuNPs at 500 mg/L; t-AuNPs at 1000 mg/L; and SWNTs at 200 mg/L. To facilitate suspension of SWNTs, 0.17% (w/w) Pluronic F108 was added to these samples.

The conductivity of all nanoparticle suspensions was less than 3 mS/m. At 100 W generator power, the heating rates of such low conductivity solutions are too slow (<0.01° C./s) to be measured accurately. By increasing the generator power to 900 W, Applicants reproducibly measured the heating rates of nanoparticle suspensions from 23° C. to 27° C. or up to 60 seconds, as described previously. Using the heating rate model fit described previously, Applicants estimate Eₐₚₚ at this power to be 500 kV/m, which accounts for maximum heating rates (˜4.0° C./s) observed at 900 W (data not shown).

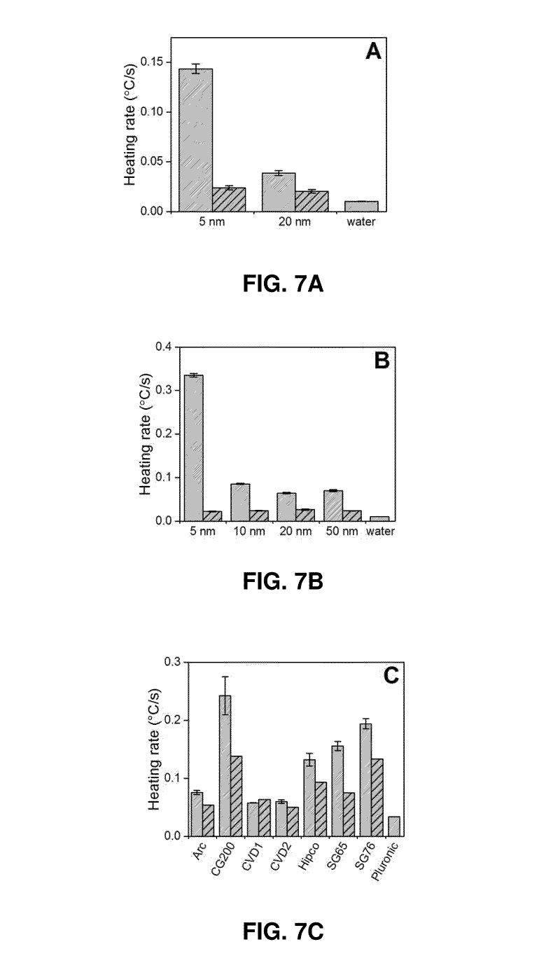

FIGS. 7A-C show the heating rates of each nanoparticle type and size. Following RF exposure, SWNTs and AuNPs were centrifuged through a 50 kDa filter, and the supernatant was then re-exposed to the RF field. With this procedure, Applicants show that the presence of the nanoparticles contributes to RF heating.

Applicants found nanoparticle heating rates to be equal to those of NaCl solutions (0-14.0 ppm) of similar conductivity (FIG. 6B). This direct comparison to salt solutions, which requires thorough nanoparticle purification as well as high RF power to observe heating, provides a better understanding of nanoparticle heating.

Like electrolyte solutions, nanoparticle suspensions fall on the aqueous heating curve. With this data, Applicants conclude that the nanoparticles themselves do not significantly contribute to bulk heating by any special heating mechanism. Instead, the charge of the nanoparticles contributes to the overall conductivity of the system, which heats via dielectric loss.

The SWNT conductivities (0.35-1.18 mS/m) were less than that of a 7.0 ppm NaCl solution. Measurable differences in conductivity can be due to minute differences between samples, such as extent of nanotube functionalization and even residual catalyst, despite extensive purification efforts. The high SWNT concentration produced opaque samples that precluded the observation of any alignment effects, such as those observed for m/s-SWNTs.

The 5 nm c-AuNPs and t-AuNPs produced greater heating than their larger counterparts. Previously, an electrophoretic effect was proposed to account for excess heating by small AuNPs. However, these calculations did not adequately account for differences in the effective electric field of the nanoparticle suspensions. Applicants envision that, because smaller AuNPs have a greater surface area to mass ratio, the total surface charge present in the 5 nm AuNP suspensions is greater than that of larger AuNPs.

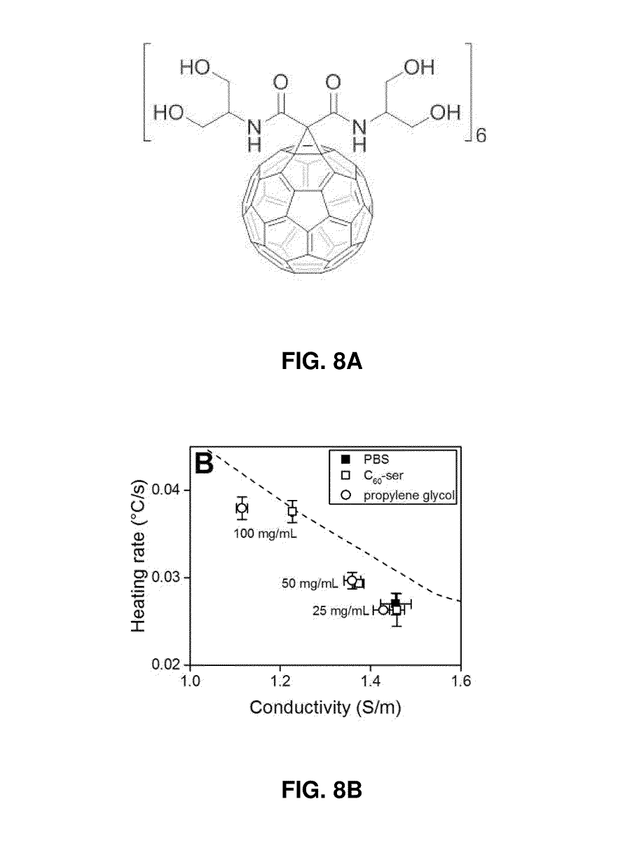

Additionally, the centrifugation-based purifying procedure cannot remove all contaminant counterions due to strong electrostatic attractions between the counterions and the nanoparticle surface. Nanoparticles, however, could be functionalized to have water-structuring activity like the kosmotropic materials presented above. Co-ser, a neutral, water-soluble fullerene derivative shown in FIG. 8A, was prepared according to the literature procedures (Bioorg. Med. Chem. 10, 3545-3554 (2002); and Journal of Chromatography A. 1169, 86-94 (2007)). C₆₀-ser reduces the conductivity of PBS and enhances its heating rate accordingly like the kosmotropic materials described previously. To Applicants' knowledge, this is the first time a nanoparticle has been used to enhance the bulk heating rate of a highly conductive, biologically relevant system.

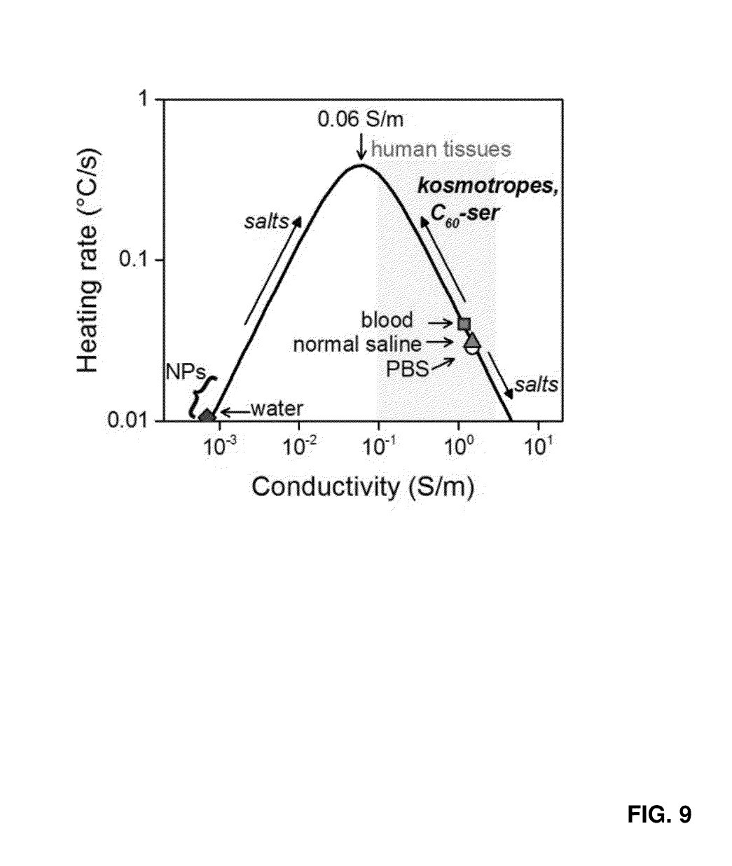

Since gold colloids were first reported to produce heat upon exposure to a RF electric field, numerous conflicting studies on nanoparticle RF heating behavior have been published. Prior studies in vitro have characterized the heating behavior of saline solutions alone. Here, Applicants have generalized that heating model to a diverse assortment of salts and shown that the RF heating behavior is determined by the dielectric and thermal properties of the bulk solution independent of the atomic or molecular identity of the salt, with peak heating occurring at a bulk conductivity of 0.06 S/m at 13.56 MHz. Furthermore, nanoparticles including cAuNPs, tAuNPs, and SWNTs heat like salt solutions of the same conductivity and can also be found on the heating curve (FIG. 9). These results indicate that these materials contribute to RF heating only by altering the bulk conductivity of the medium.

At 13.56 MHz, the conductivity of biological systems exceed the peak heating conductivity found for aqueous systems, precluding the practice of adding more salts or polar materials for heating rate enhancement. This addition actually reduces the heating rate of the medium.

### Example 1.5

### Sample Preparation

Aqueous salt solutions were prepared in high-resistivity water (18.2 MΩ/cm) using a number of binary salts (NaCl, NaC₂H₃O₂, MgCl₂, Na₂SO₄, AuCl₃, Na₃C₆H₅O₇) at concentrations varying from 0.01 mM to 200 mM. For kosmotropic material experiments, 1 mL of concentrated PBS containing 90 g/L NaCl, 1.44 g/L KH₂PO₄, and 7.95 g/L Na₂HPO₄ was added to a known mass of kosmotropic material, followed by dilution to 10 mL to ensure equal ion concentration across all samples. Kosmotropic materials (i.e., sucrose, maltose, glucose, glycerol, propylene glycol, ethylene glycol, sorbitol, glycine, sarcosine, glycine betaine, and dimethylsulfoxide) and all salts were obtained from Sigma-Aldrich in high purity (>98%) and used as received.

### Example 1.6

### Nanoparticle Purification

Citrate-capped gold nanoparticles (5, 10, 20 nm) were obtained from Ted Pella, Inc. in ˜0.05 mg/L stock solutions. Tannic acid-capped gold nanoparticles (Ted Pella, Inc.) that were extensively washed were obtained completely free of contaminants (<0.000001 trace elements) from the manufacturer. Stock solutions were washed and concentrated using 50 kDa centrifuge filters. Short filtration at high speeds (125 s, 3500 rpm) removes ˜70% of the solution. Nanoparticles are washed at least nine times until the resulting supernatant has dielectric and RF heating properties identical to those of pure water.

### Example 1.7

### Nanoparticle Characterization

SWNTs were analyzed by Raman spectroscopy and X-ray Photoelectron Spectroscopy. Raman spectra were obtained on a Renishaw inVia Raman microscope with excitation at 514 nm. XPS samples were prepared by pressing onto indium foil. Data was collected on a Phi Quantera SXM system.

### Example 1.8

### Electrical Characterization

Complex permittivity measurements were taken using an Agilent 85070E high-temperature coaxial dielectric probe (Agilent Technologies, Santa Clara, Calif.) connected to an Agilent E4991A impedance analyzer to extend the frequency range of the probe down to 10 MHz. Four-point calibration (open, short, 50 ohm load, low-loss capacitor) of the impedance analyzer was performed prior to each measurement session. Three-point probe calibration of the probe (air, short, water) was performed periodically between samples. A single-point calibration (air) was performed between every sample to eliminate signal drift. For measurements, 600 μL of sample was loaded onto the probe and approximately 400 logarithmic data points were acquired across the frequency range of 10 MHz to 3 GHz, with each measurement performed in triplicate in rapid succession to avoid probe drift. Applicants found no difference between loading a small amount of sample onto the probe and immersing the probe into a plastic beaker containing 20 mL of sample.

Additionally, for each sample in the 900 W experiment, static solution DC conductivity was measured ten times using either an InLab 731 or 741 conductivity probe (Mettler Toledo, Columbus, Ohio), depending on the conductivity of the sample and the measurement range of each probe (data not shown). DC conductivity measurements were in agreement with low frequency AC conductivity measurements performed using the impedance analyzer.

### Example 1.9

### Radiofrequency Heating

A variable power RF (13.56 MHz) Generator System (ThermMed, LLC, Erie, Pa.) was used to heat aqueous solutions. A high-voltage RF field is generated in the 8 cm air gap between the transmitting and receiving heads of the RF field generator. For aqueous experiments, samples were placed in a 1.3 mL quartz cuvette on a non-conducting Teflon holder mounted on an adjustable rotary stage at ambient temperature and open to air. The cuvette was placed 8 mm from the transmitting head of the RF field generator and exposed to the high-voltage RF field at 100 or 900 W generator power. Sample temperatures were recorded using an infrared camera (FLIR SC 6000, FLIR Systems, Inc., Boston, Mass.) approximately every 0.17 s. At both 100 W and 900 W, samples were heated from ambient temperature (i.e., ˜23° C.) to 27° C. or for a duration of 60 seconds, whichever occurred first. Heating rates were calculated by fitting a linear regression to the temperature versus time plot of each sample for the duration of RF exposure.

### Example 1.10

### Electrode Polarization Correction in Dielectric Measurements

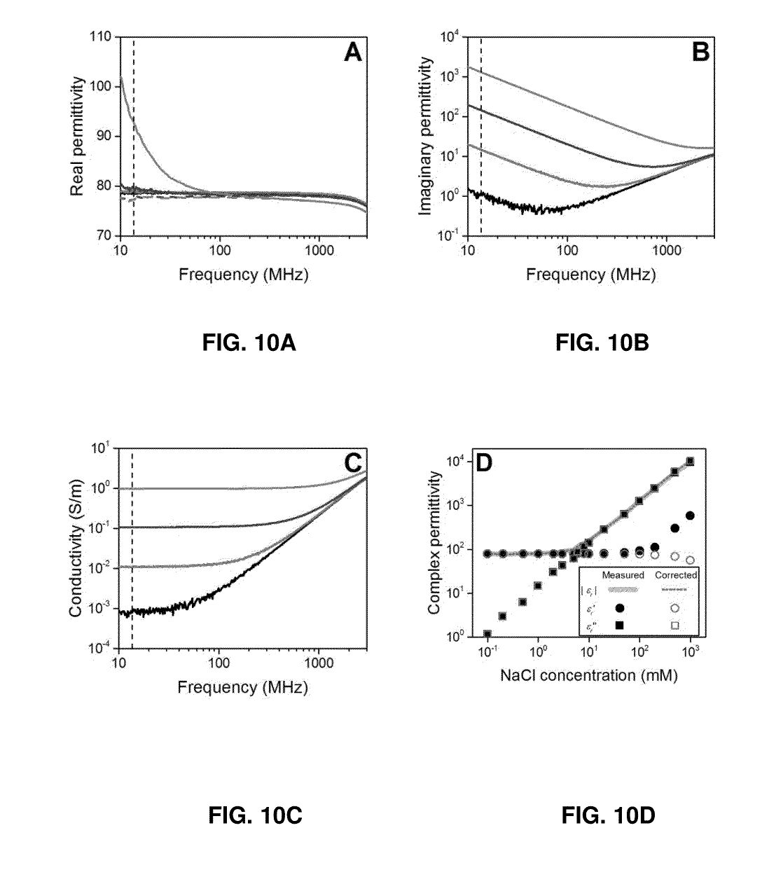

When measuring the relative permittivity of highly conductive solutions, electrode polarization occurs at low frequencies, which can result in significant instrument-related measurement errors, especially for real permittivity ∈′ᵣ. Applicants have performed the necessary corrections and found these errors to be insignificant (<1.2% for σ and ∈ᵣ) for Applicants' purposes in the conductivity range studied. The results are summarized in FIGS. 10A-D.

### Example 2

### Optimizing Radiofrequency-Induced Heating of Biological Tissues

This Example expands on the experimental results of Example 1. In this Example, Applicants describe methods of modulating the heating properties of biological tissues by using biocompatible solutions that can induce selective, targeted hyperthermia when exposed to a non-invasive radiofrequency hyperthermia (RFH) system.

The RFH system shows promise as a method for treating a multitude of cancer types at different stages of the disease. However, the main drawback is selectively targeting the cancer and not the neighboring, healthy tissues. A method to modulate (e.g., alter) the electrical properties (e.g., permittivity and conductivity) of both cancerous and healthy tissues can significantly increase the therapeutic index of this system and optimize the RFH technique. This Example describes such a method, using biocompatible, non-toxic solutions.

A theoretical model for the radio frequency (RF) heating behavior of sodium chloride (NaCl) solutions was developed and tested using a microstrip waveguide (J. Appl. Phys., 2013, 113, 074902). This model accounts for the peak RF heating of NaCl solutions that occurs at a conductivity of approximately 600 μS/cm. At higher conductivities, the difference in dielectric properties between the NaCl solution and air causes a shielding effect of the electric field. The electric field must obey the boundary condition ∈₁E₁=∈₂E₂, resulting in a reduced effective electric field within the sample.

Although the aforementioned approach accurately predicted experimental heating rates for NaCl solutions, the heating rates involved in this study are over four orders of magnitude slower than those Applicants have previously reported (up to 0.4° C./s for purified gold nanoparticles and up to 3.6° C./s for salt solutions) (J. Phys. Chem. C, 2012, 116, 24380).

### Example 2.1

### Sample Preparation

Aqueous salt solutions were prepared in high-resistivity water (18.2 MΩ/cm) using a number of monovalent (LiCl, NaCl, CsCl, NaI, NaOAc), divalent (MgCl₂, CaCl₂, Na₂CO₃, Na₂SO₄), and trivalent salts (FeCl₃, AuCl₃, Na₃PO₄, Na₃(C₆H₅O₇) (sodium citrate)) at concentrations varying from 0.01 mM to 1.5 M.

Kosmotropic material solutions in phosphate buffered saline (PBS) were prepared using concentrated PBS to maintain equal ion concentration in all solutions. Kosmotropic materials (sucrose, maltose, trehalose, glycerol, propylene glycol, ethylene glycol) and all salts were obtained from Sigma-Aldrich in high purity (>98%) and used as received.

### Example 2.2

### Electrical Characterization

Static solution DC conductivity was measured ten times per sample using an InLab 731 conductivity probe (Mettler Toledo, Columbus, Ohio). Complex permittivity measurements were taken using an Agilent 85070E high-temperature coaxial dielectric probe (Agilent Technologies, Santa Clara, Calif.) connected to an Agilent E4991A impedance analyzer to extend the frequency range of the probe down to 10 MHz. Approximately 400 logarithmic data points were taken across the frequency range of 10 MHz to 3 GHz with each measurement performed in triplicate.

### Example 2.3

### Radio-Frequency Heating System

A variable power Kanzius External RF system (13.56 MHz) Generator System (ThermMed, LLC, Erie, Pa.) was used to heat aqueous solutions and tissue samples. A high-voltage RF field is generated in the 8 cm air gap between the transmitting and receiving heads of the RF-field generator. For aqueous experiments, samples were placed in a 1.3 mL quartz cuvette on a nonconducting Teflon holder mounted on an adjustable rotary stage at ambient temperature and open to air. The cuvette was placed 8 mm from the transmitting head of the RF-field generator and exposed to the high-voltage RF field at 900 W generator power. Sample temperatures were recorded using an infrared camera (FLIR SC 6000, FLIR Systems, Inc., Boston, Mass.) approximately every 0.17 seconds. Samples were heated from ambient temperature (i.e., ˜23° C. to ˜70° C. or for 120 seconds), whichever occurred first. Heating rates were calculated by fitting a linear regression to the temperature versus time plot of each sample for the duration of RF exposure or only for the first five seconds of RF exposure. The latter methodology allows for the assumption that the dielectric properties remain approximately constant in the time frame analyzed, as discussed herein.

Similar experiments were performed for NaCl and MgCl₂ at 100 W. The solutions were heated until the sample temperature reached 27° C. or for 120 seconds, whichever occurred first. In this small temperature range, the temperature-dependent dielectric properties that determine RF heating rate can be assumed to be relatively constant.

### Example 2.4

### RF Heating of Aqueous Solutions

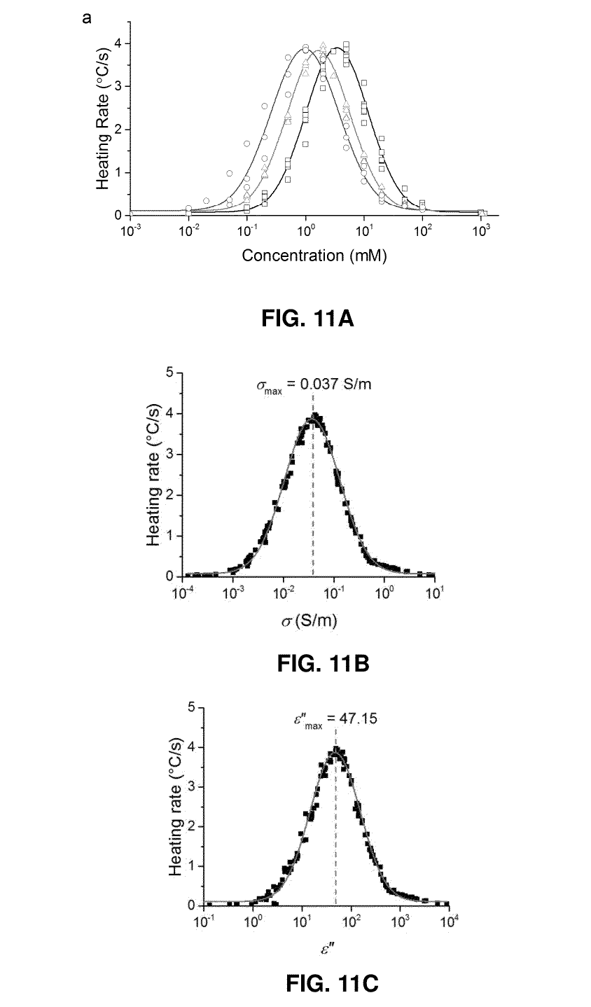

A number of aqueous salt solutions prepared in ultra-pure water were exposed to a 13.56 MHz RF field. Exposure at 900 W lasted for 120 seconds or until the solution temperature reached 70° C. The RF heating rates of the solutions were calculated by fitting a least-squares regression to the temperature versus time plot of each sample for the duration of RF exposure. The heating rates of all salt solutions, regardless of valence, generate a bell-shaped heating curve as a function of concentration on a log scale (FIG. 11A). On average, peak heating occurred at 3.46, 1.67, and 1.28 mM for mono-, di-, and trivalent salts, respectively.

At a fixed concentration, salts of greater valence are more conductive than salts of lesser valence because a greater number of ions are present. This observation led Applicants to compare the heating rates of different salt solutions as a function of their dielectric properties. The static DC conductivities of all solutions was measured as well as the complex permittivity in the range of 10 MHz to 3 GHz. Given the single operating frequency of the RF generator, only the ∈′ and ∈″ values at 13.56 MHz are considered here.

The three distinct heating curves for mono-, di-, and trivalent salt solutions collapse into a single curve when the dielectric properties of the solutions are considered rather than concentration. FIGS. 11B-C show that peak heating occurs at σ=0.037 S/m or ∈″=47.15. Conductivity (σ) may be used interchangeably with imaginary permittivity (∈″) using the relationship σ=∈″∈₀ω, where ∈₀ is the permittivity of free space and ω is the angular frequency ω=2πf, and f is the frequency. In the experimental data to date, f is 13.56 MHz. Moreover, at high power, the temperature range considered varies greatly between slow-heating (˜23-25° C.) and fast-heating solutions (˜23-70° C.).

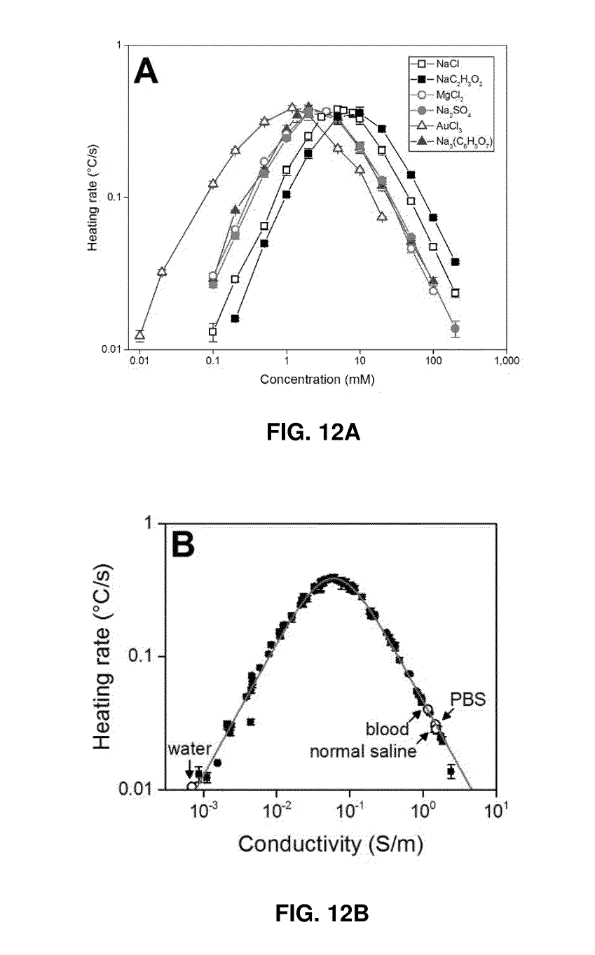

In similar experiments, different salt solutions were heated under RF exposure at 100 W from ambient temperature (˜23° C./s) up to 27° C. or for 120 seconds, whichever occurred first. At this lower power, the temperature range considered is approximately the same for all solutions, regardless of heating rate. FIGS. 12A-B show the RF heating rate of these salt solutions at low power as a function of concentration, conductivity, and imaginary permittivity. The peak heating values for conductivity and imaginary permittivity at 100 W are σ=0.0589 S/m and ∈″=78.0, respectively. These values are in great agreement with prior results, even though the heating rates presented here are over three orders of magnitude greater.

At 900 W, solutions heat more quickly and reach higher temperatures. In these circumstances, heating rates are affected by the temperature-dependence of the solutions' dielectric properties and additional heating mechanisms of conduction and convection. These results suggest that ionic solutions heat according to the dielectric properties of the bulk solution, regardless of solute identity, even when heated to high temperatures for extended periods of time.

### Example 2.5

### Modeling Salt Heating Behavior

A model can be used to predict the RF-induced heating of aqueous solutions based on their dielectric properties. The heating rate of a material in an electromagnetic field is given by Equation 4.

$$HR = \frac{\partial T}{\partial t} = \frac{P}{ρ {c}_{p} {V}_{s}} \qquad ( 4 )$$

In Equation 4, P is the power absorbed by the material, ρ is the density, cₚ is the specific heat capacity, and Vₛ is the volume. The power absorbed is related to the material's dielectric properties by Equation 5.

$$P = \frac{{ωɛ}_{0} {ɛ}^{″} {\vert {E}_{eff} \vert}^{2} {V}_{s}}{2} \qquad ( 5 )$$

In Equation 5, w is the angular frequency of the field, ∈₀ is the permittivity of free space, ∈″ is the material's imaginary permittivity, and E_{eff} is the strength of the effective electric field within the material. When an electric field meets the interface between two materials of different dielectric properties, such as the interface between air and an aqueous solution in Applicants' experiments, it generally obeys the boundary condition illustrated in Equation 6.

∈_{r,1}E₁=∈_{r,2}E₂ (6)

In Equation 6, ∈ᵣ=∈′+i∈″ is the relative permittivity, which has real (∈′) and imaginary (∈″) components. This boundary condition is modified to take into account the geometry of the materials. In Applicants' system, the sample shape can be approximated as a prolate spheroid with a=b=5 mm and c=9 mm. The effective electric field for a material contained in a dielectric medium (air) is given by Equation 3 from Example 1 and reproduced herein.

$${E}_{eff} = \frac{1}{1 + N ( \frac{{ɛ}_{r}}{{ɛ}_{r , air}} - 1 )} {E}_{app} \qquad ( 3 )$$

In Equation 3, N is the polarization tensor. Improved calculation for N is given in example 1, where the cuvette is approximated as a cylinder rather than a prolate spheroid. The polarization tensor, along the major axis of a prolate spheroid, which in Applicants' case is aligned with the electric field, is characterized by Equation 7.

$${N}_{c} = \frac{1 - {e}_{p}^{2}}{2 {e}_{p}^{3}} ln ( \frac{1 + {e}_{p}}{1 - {e}_{p}} - 2 {e}_{p} ) \qquad ( 7 )$$

In Equation 7, eₚ=\sqrt{1−a²/c²}. In this model, all variables can be accurately measured, or approximated in the case of N, with the exception of Eₐₚₚ. Given the high voltages involved, the electric field in air cannot be measured with any known instrumentation due to immediate overheating upon exposure to the RF field. As a result, this parameter must be fit to the heating rate data obtained.

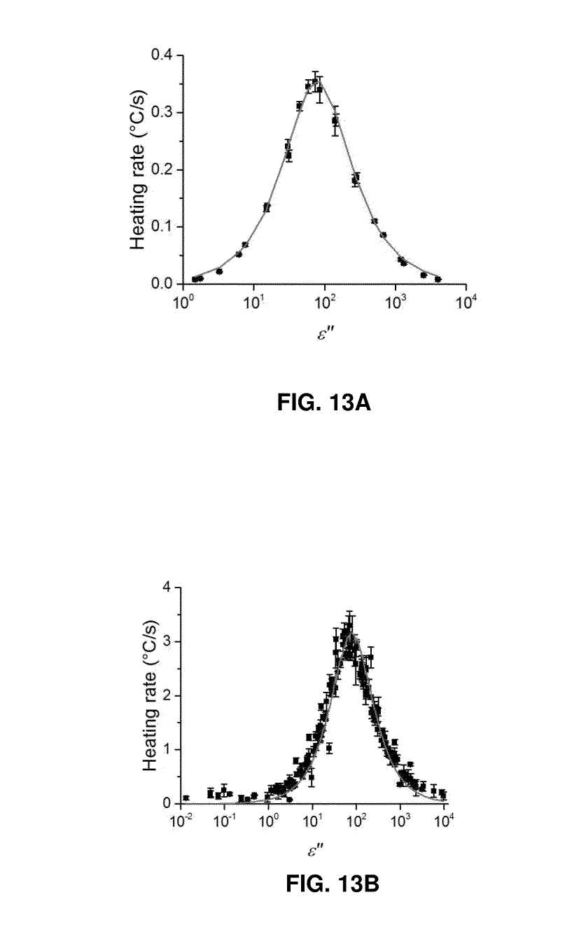

Regardless of the value for Eₐₚₚ, peak heating is expected to occur at ∈″=78.7, which is in agreement with Applicants' experimental data at 100 W (Eₐₚₚ=465±3 kV/m, FIG. 13A), but not at 900 W, as shown in FIGS. 12A-B and 11C, respectively. This is unsurprising given the wide temperature range considered for 900 W experiments as noted above.

If only the first five seconds of RF exposure are considered in calculating heating rate, however, peak heating at 900 W occurs at ∈″=81.3 (Eₐₚₚ=1.390±7 MV/m), as shown in FIG. 13B. At this high power, initial instantaneous heating rates, especially for fast-heating solutions, may vary greatly for a single sample because a limited number of data points are available.

Values calculated for Eₐₚₚ seem large. Given the large wavelengths (22 m) corresponding to a 13.56 MHz field and the proximity of Applicants' samples to the RF field source, these experiments are conducted in the reactive near field, where an enhancement of the heating rates could be due to near field effects.

Enhanced heating could also result from current generated within the sample due to the magnetic field. Moreover, local induced fields within the sample may have an amplifying effect on the effective electric field. As a result, the calculated values of Eₐₚₚ are possibly overestimated, since they account for all of these possible heating mechanisms.

Regardless, experimental results and the model presented here show that the RF heating properties of aqueous salt solutions are dependent on their dielectric properties and thermal properties.

### Example 2.6

### Modulating Salt Solution Properties to Optimize RF-Induced Heating

Applicants have shown that the RF heating properties of salt solutions, regardless of salt identity, depend on the properties of the bulk solution, including permittivity, heat capacity and density. Heating rate increases approximately linearly with imaginary permittivity (∈″), reaching a peak around ∈″=78.7 or σ=0.06 S/m, beyond which heating rate decreases steadily. The decrease in heating is the result of a diminished effective electric field within the sample due to the large difference in dielectric properties between the sample and host medium. Unfortunately, biologically-relevant systems (e.g. blood, intra- and extra-cellular space) have high ion content and their imaginary permittivities exceed this peak. The addition of ionic salts or even polar materials to such a system can only increase permittivity, and thus attenuate RF heating.

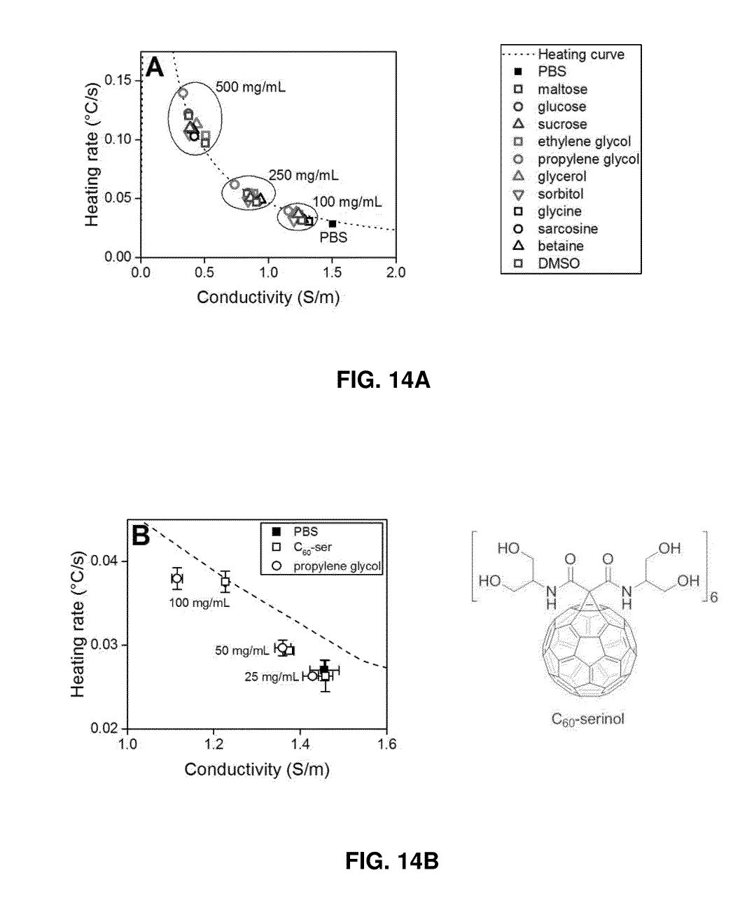

The aforementioned effect can be utilized to protect healthy tissue from excessive RF heating. However, enhancing the heating in cancerous tissue presents a bigger difficulty. Based on Applicants' model, it is also clear that this challenge can be overcome by adding a material of low heat capacity (cₚ) compared to that of water, such as ethanol (FIG. 14C). Clinically, such an approach could be coupled with chemical embolization therapy.

However, most salts have low solubility in high ethanol concentrations. Moreover, neat ethanol heats poorly in the RF field (0.09° C./s at 900 W). Other materials that decrease the heat capacity of biologically relevant solutions remain to be tested.

Another property that could be adjusted is density (ρ). However, practically decreasing the density of an aqueous system significantly would be very difficult.

A preferred approach for enhancing the heating rate of biologically-relevant solutions may be to decrease their conductivity using kosmotropic materials (as discussed in Example 1). Applicants measured the RF heating and dielectric properties of a number of kosmotropic materials dissolved in phosphate buffered saline (PBS), a biologically relevant solution with conductivity (a) of 1.46 S/m. At high concentrations (685 mg/mL), the RF heating rate of PBS was increased for all kosmotropic materials tested (FIG. 14A). Maltose and trehalose concentration was limited to 342 mg/mL due to lower solubility, but still enhanced PBS heating rate to 0.32±0.01 and 0.33±0.01° C./s, respectively, at this concentration.

A concentration-dependent study (0-10 M) was completed for the glycerol, propylene glycol, and ethylene glycol in PBS (FIG. 14B). Increasing kosmotropic materials concentration lead to a decrease in conductivity and a corresponding increase in RF heating rate, except at 10 M glycerol and propylene glycol, where heating rate deviated from the salt heating curve. This deviation could possibly result from the low water content in the solutions, approximately 26% (v/v), which prohibits approximating the solutions as being aqueous.

### Example 2.7

### Modulating the Conductivity and RF Heating Rate of Blood

Previous experiments involved conductivity modulation of PBS, a biologically relevant aqueous solution commonly used in laboratory settings. Using bovine blood, Applicants demonstrate the biological application of conductivity modulation for optimization of RF heating. Blood samples contained 2-3% ethylenediamine tetraacetic acid (EDTA) to prevent coagulation. A number of additives, including water, normal saline, propylene glycol and glycerol were mixed with blood at increasing weight percent content (1-20%).

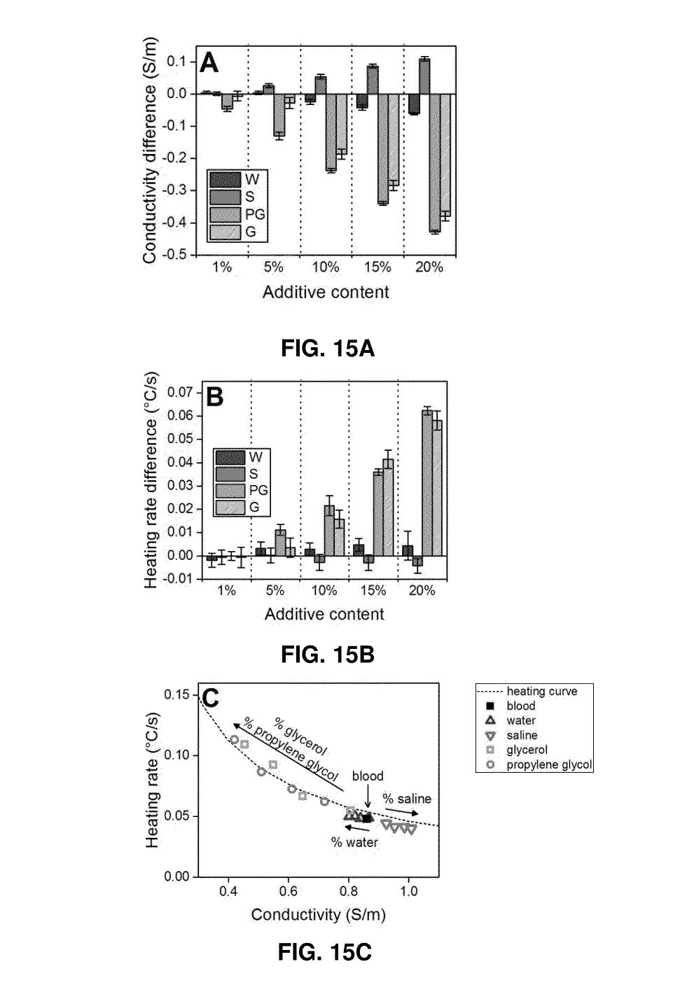

FIGS. 15A-B show the difference in conductivity and heating rate between blood plus additive and blood alone. Addition of normal saline, which has a greater conductivity than blood, increases the total ionic content of the mixture, thereby increasing the conductivity and decreasing the heating rate. Propylene glycol and glycerol have the opposite effect, as illustrated by the bars which point in opposite directions. Water results in similar effects as propylene glycol and glycerol. However, the changes are not statistically significant. FIG. 15C is a different representation of FIGS. 15A-B that clearly illustrate the ability to move in both directions along the RF heating curve by addition of different materials.

### Example 2.8

### Modulating Real Permittivity

Thus far, Applicants have demonstrated the ability to optimize RF heating rate by modulating the conductivity of a material. Theoretical calculations, however, suggest that reducing real permittivity ∈′ could result in RF heating greater than what can be achieved in purely aqueous systems.

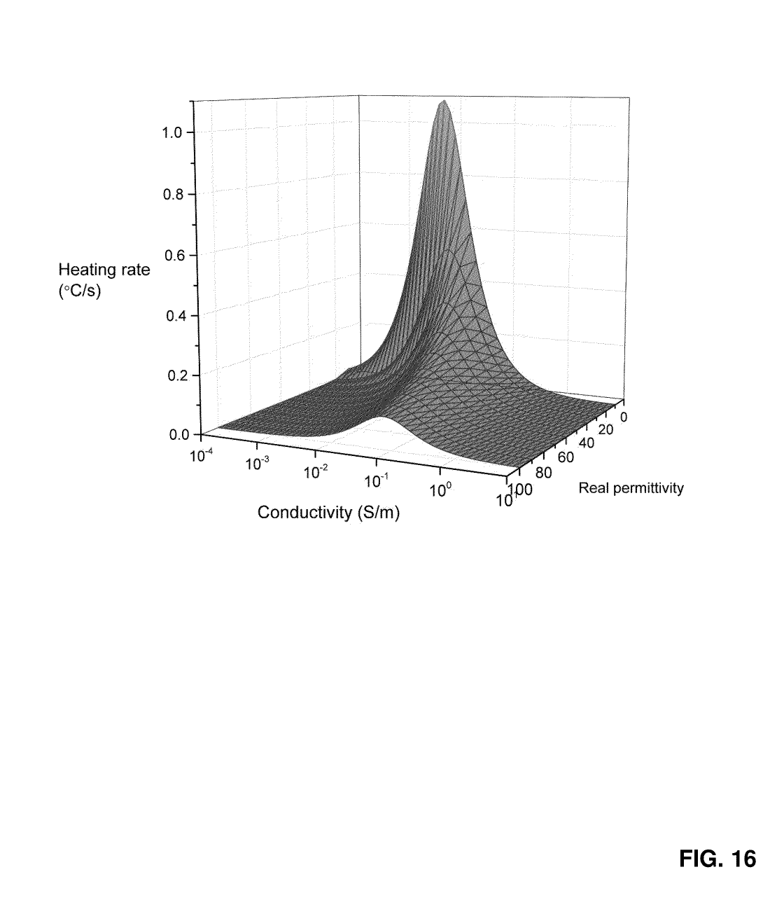

The three-dimensional plot in FIG. 16 shows heating rate with respect to conductivity (σ) and ∈′ (assuming all other properties are constant). Peak heating occurs when real and imaginary permittivity are equal (∈″=∈′ or σ=∈′∈₀ω). Therefore, as ∈′ decreases, so does the conductivity at which peak heating occurs as well as the maximum possible heating rate. These results help explain why fatty tissue heats faster than non-fatty tissue under RF exposure and point towards materials that may enhance heating rate.

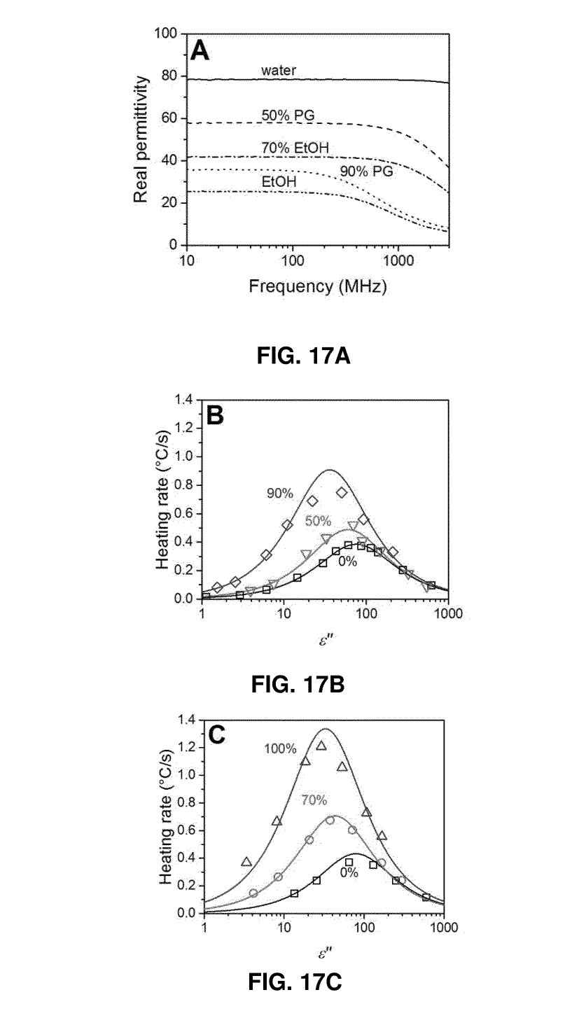

FIGS. 17A-C show experimental results supporting the theoretical calculations of FIG. 16. Materials of different real permittivities (FIG. 17A) were used to generate heating curves similar to the aqueous heating curves in FIGS. 12A-B. Mixtures of water, ethanol (EtOH) and propylene glycol (PG) were used to prepare these materials: water, EtOH, 70% EtOH, 50% PG, and 90% PG. NaCl or LiCl were added to these materials in increasing concentrations to increase ∈″ (conductivity). FIGS. 17B-C show that, as ∈′ decreases, peak heating occurs at lower ∈″ and the maximum peak heating rate also increases.

The aforementioned results show that, as the real permittivity of a material decreases, its maximum RF heating rate increases. The aforementioned observations are useful for heating rate modulation because it suggests that heating rate enhancement of cancerous tissue could be achieved by addition of low real permittivity materials, such as propylene glycol and ethanol, as well as fats, lipids, vitamins and oils.

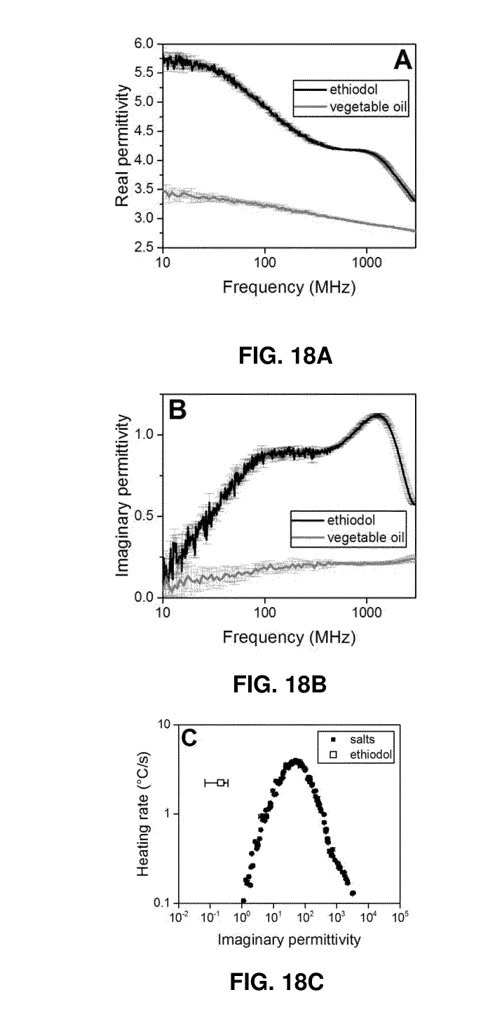

FIGS. 18A-C show the dielectric properties (∈′ and ∈″) of ethiodol, a drug commonly used in chemoembolization applications as a contrast agent, and store-bought vegetable oil. These are examples of low real permittivity materials that show enhanced RF heating compared to aqueous systems of the same conductivity (FIG. 18C). Modulation of ethiodol ∈″ using conductivity enhancing materials would lead to even greater RF heating.

In this Example, the RF heating behavior of salt solutions, regardless of identity, has been attributed to the dielectric and thermal properties of the bulk solution. A model fit for calculating heating rates from these properties has been developed and applied even to very fast heating solutions. Diminished heating rates for solutions with imaginary permittivity exceeding ∈″=78.7 (σ=0.067 S/m) result from the boundary condition obeyed by electric field at the interface of two dielectric materials, reducing the effective electric field within the solution.

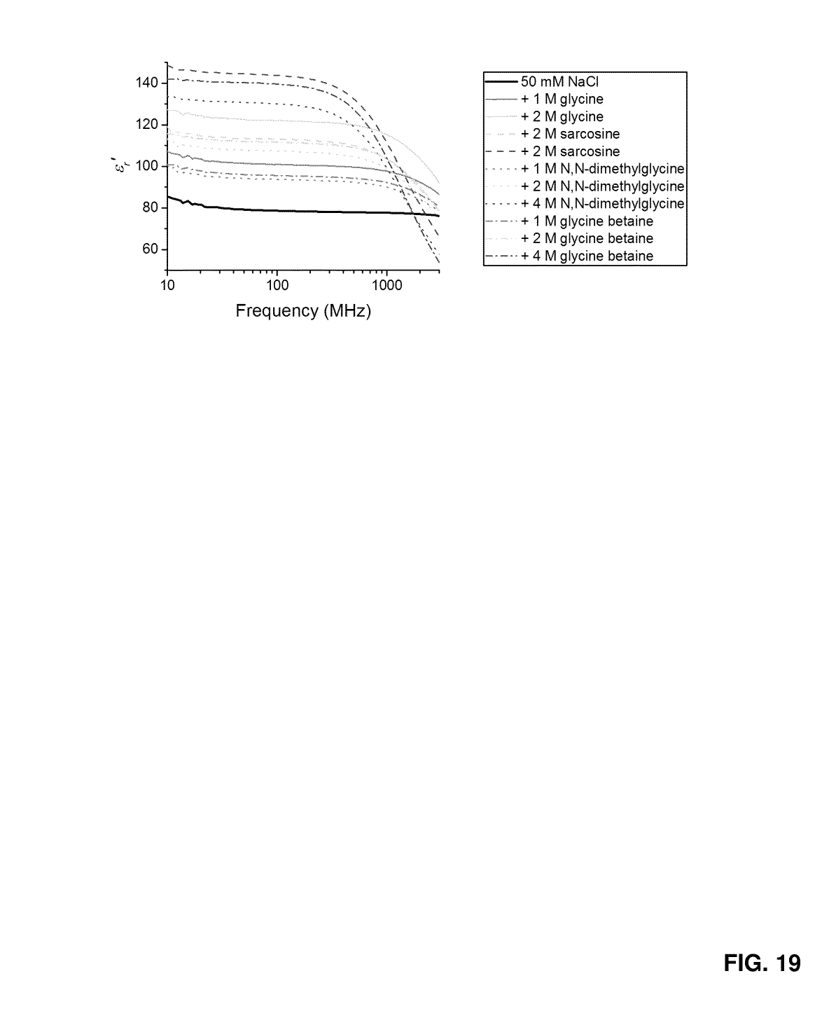

The dielectric properties of biological systems exceed these peak values, precluding the use of salts or polar materials for enhancing RF heating of these systems. Instead, the heat capacity or permittivity of biologically-relevant solutions can be reduced to increase heating rate accordingly, as is predicted by the model described herein. Low concentrations of ethanol or other materials with low heat capacity can be introduced to enhance heating rate without greatly diminishing salt solubility. It is also possible to increase ∈′ to increase the peak heating conductivity and reduce the maximum possible heating rate using highly polar materials such as zwitterions, including glycine betaine, sarcosine, glycine, and N,N-dimethylglycine (FIG. 19).

Importantly, the ability of kosmotropic materials to stabilize water structure can be exploited to reduce ion mobility and conductivity and enhance RF heating rate accordingly. With the exception of ethylene glycol, many of the substances tested here for enhancement of RF heating are considered safe when ingested in moderate quantities and are widely available, making them highly attractive for clinical use.

### Example 3

### Radiofrequency Electric-Field Heating Behaviors of Highly Enriched Semiconducting and Metallic Single-Walled Carbon Nanotubes

In this Example, Applicants demonstrate that aqueous suspensions of metallic and semiconducting single-walled carbon nanotubes produce high heating rates when exposed to radiofrequency (13.56 MHz) electric fields. Heating was examined as a function of host medium conductivity, which was modulated using NaCl. These results can aid in the development of heating agents for non-invasive radiofrequency cancer hyperthermia.

In particular, Applicants examined in this Example the RF-induced (13.56 MHz) heating behaviors of 95% metallic- and semiconducting-enriched single-walled carbon nanotubes (m-/s-SWNT) suspended in aqueous solutions of varying NaCl molarity (0.001 mM-1 M). Heating effects were only evident for host molarities below 1 mM (equivalent to 0.1 S/m), whereby s-SWNT heating rates dominated that of m-SWNTs.

The heating effects were localized to aligned and aggregated ‘SWNT-ropes’ of length ˜1 cm that form in suspension, parallel to the electric-field vector, during RF exposure. For molarities above 1 mM, no enhancements were evident due to the large heating effects of the bulk ionic NaCl suspensions, which have been observed in previous studies. Although larger enhancement effects proportional to host conductivity have been theoretically predicted for m-/s-SWNT suspensions, this was not observed and is most likely due to aggregation and screening effects, which diminish the scattered electric field in close vicinity to the m-/s-SWNTs.

### Example 3.1

### Sample Preparation and Purification

Using RF experimental setups described previously (J Phys Chem C Nanomater Interfaces 2012, 116, 24380-24389), Applicants investigated the RF-induced heating rates of highly-enriched (95%) full-length (300 nm to 2 μm) m-/s-SWNTs as a function of SWNT concentration (1-100 mg/L) and host conductivity (DI water or NaCl solutions ranging from 10⁻⁴ to 10³ mM). All solutions were dispersed in a 2% Pluronic F108 surfactant to minimize aggregation as much as possible.

Highly purified and enriched samples (in powder thin-film form) were purchased from NanoIntegris (Quebec, Canada) and gently sonicated in DI water (18.2 MΩ/cm) until the thin sheets of nanotubes had been broken up. The samples were then washed several times through 100 kDa filters to remove any surfactant or ionic contaminants. Ultra high-speed centrifugation (55,000 g) was then utilized to force the SWNTs out of suspension so that aliquots of the background supernatant could be isolated and tested in the RF field against DI water heating rates. When the two heating rates were equivalent, the SWNT sample was deemed fully purified from ionic contaminants, such as iodixanol, which is in the SWNT separation process as part of the density gradient medium. Samples were then lyophilized and weighed using a microbalance to create aqueous m-/s-SWNT suspensions (in 2% w/v Pluronic F108) of concentrations 100, 50, 25, 10, and 1 mg/L. The 100 mg/L suspension was found to be the upper limit concentration that would produce fully stable, minimally-aggregated SWNTs.

### Example 3.2

### Sample Characterization

Purified m-/s-SWNT samples were characterized via Raman spectroscopy, ultraviolet-visible spectroscopy (UV-VIS), atomic force microscopy (AFM), scanning electron microscopy (SEM), and X-ray photoelectron spectroscopy (XPS). Raman was taken on a Renishaw 1000 micro-Raman system with a 795 nm excitation wavelength. For UV-VIS, an Agilent 8435 UV-VIS spectrometer (Santa Clara, Calif., USA) took a spectrum across the wavelength range 200 nm to 1800 nm, using 1 nm increments, with 20 second acquisition time using a quartz cuvette.

For AFM studies, aqueous solutions of the samples were drop-casted onto freshly cleaved mica and placed in a desiccator for 24 hours prior to imaging. Tapping-mode AFM images were taken in air under ambient conditions on a Digital Instruments Nanoscope IIIA (Digital Instruments, Tonawanda, N.Y., USA). SEM images were taken on an FEI Quanta 400 ESEM FEG (Hillsboro, Oreg., USA) operating at 10 kV. XPS data was obtained via a physical electronics (PHI Quantera, Chanhassen, Minn., USA) XPS/ESCA system at 5×10⁻⁹ Torr base pressure. A monochromatic Al X-ray source at 100 W was used with a pass energy of 26 eV and a 45° take off angle. The beam diameter was 100.0 μm.

### Example 3.3

### RF Electric-Field Exposure System

Once characterized, these m-/s-SWNT samples were then exposed to a high-intensity (˜90 kV/m) 13.56 MHz RF electric field. Protocols and conditions are identical to those stated previously (J Phys Chem C Nanomater Interfaces 2012, 116, 24380-24389). Approximately 1.3 mL of each sample was placed in a cylindrical quartz cuvette (˜1 cm height by ˜1 cm diameter) and exposed to the RF field for 120 seconds or until the temperature reached 70° C. to prevent electrical arcing. Thermal imaging data was captured using an FLIR SC 600 infrared camera. Videos were also recorded simultaneously using a Nikon SLR camera. Once the samples had been exposed, the SWNTs were removed from solution using a 100 KDa filter to collect the supernatant, which was then re-exposed to the RF field. The differential heating rates determined heat production from the SWNTs themselves and not the Pluronic F108/NaCl background.

### Example 3.4

### m-/s-SWNT Characterization

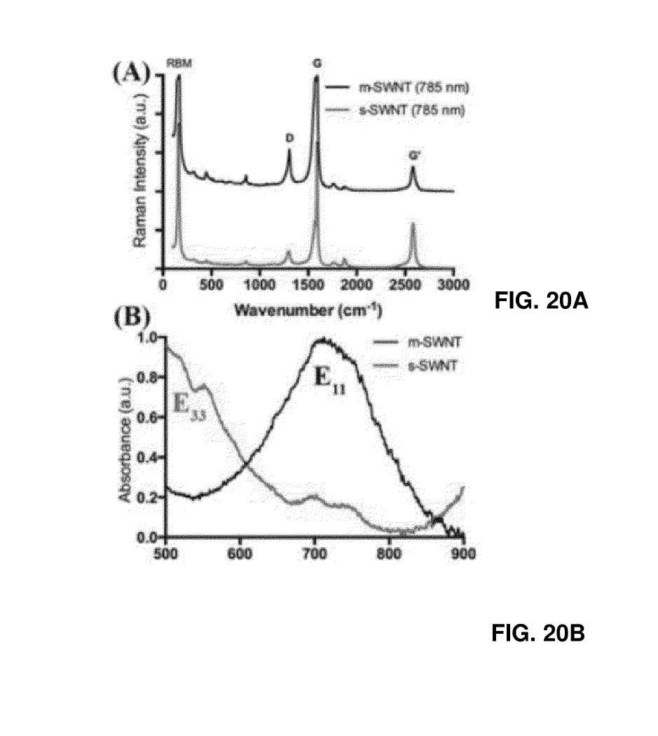

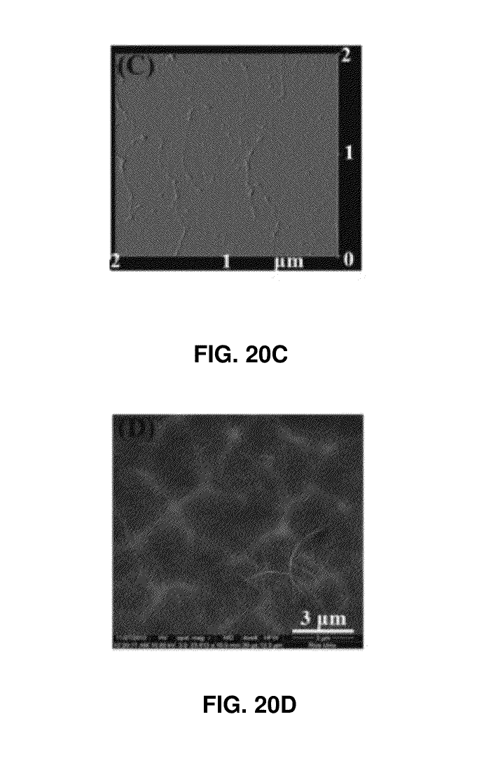

FIGS. 20A-D show the m-/s-SWNT characterization data. Raman data (FIG. 20A) depicts the radial breathing modes (RBM) as well as the D, G and G′ bands when excited with a 785 nm laser. These peak positions coincide well with previous established SWNT characterization data. FIG. 20B depicts the UV-VIS spectrum of both SWNT types. The quasi one-dimensionality of SWNTs gives rise to sharp Van Hove peak transitions in the density of electronic states. This influences the optical properties in that they are dominated by transitions between corresponding Van Hove peaks on opposite sides of the Fermi level. The lowest energy Van Hove transition (E11) for the m-SWNTs can be seen by the broad peak centered at around ˜710 nm, while the s-SWNT peaks across the range 500-570 nm are the E33 Van Hove transitions.

Both AFM and SEM revealed the samples to be similar to that stated by the supplier. Both samples were composed of SWNTs with lengths ranging from 300 nm to 2 μm, and with diameters ranging from 1 nm to 3 nm (due to the Pluronic F108 coating). XPS was also used to verify negligible level of iodine (˜0.2-0.3%) from the iodixanol density gradient medium.

### Example 3.5

### m-/s-SWNT Heating Behaviors in Water

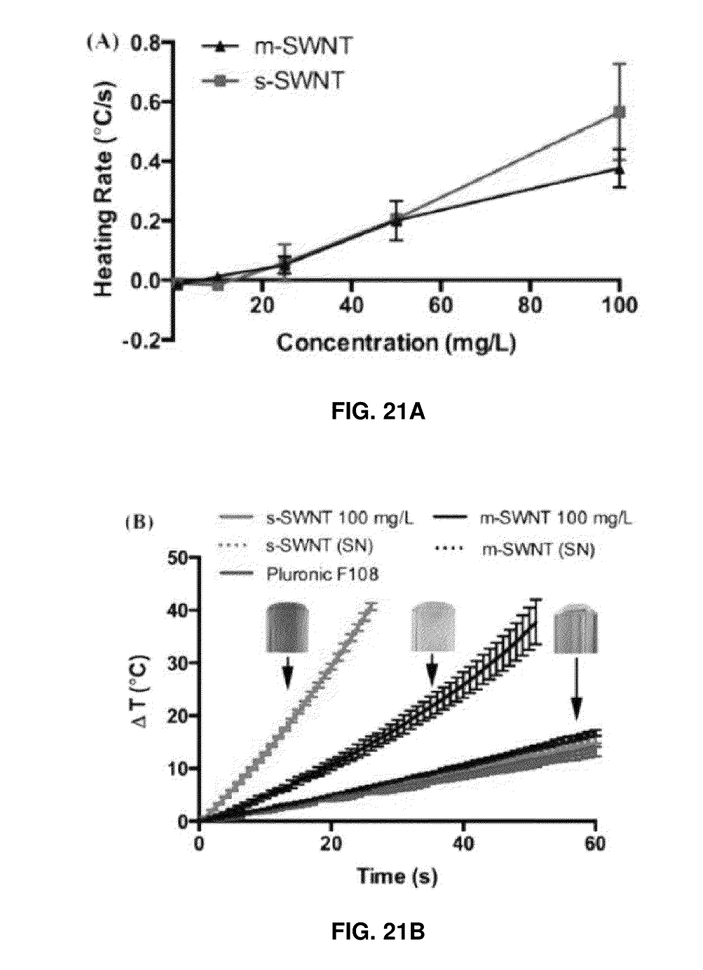

FIG. 21A depicts the derived heating rates of aqueous m-/s-SWNT Pluronic suspensions as a function of SWNT concentration, with no NaCl addition. These heating rates illustrate heat production from the SWNTs after the background supernatant heating rates have been subtracted. Heat production is only evident at a minimum concentration of 25 mg/L. There is a noticeable difference in heating rates between s-SWNTs and m-SWNTs for the highest concentration of 100 mg/L, which was found to be ˜0.55 and ˜0.36° C./s, respectively.

When compared to the heating rates of charged citrate-capped AuNPs with diameters of 5 nm when exposed to similar strength electric fields, s-SWNTs and m-SWNTs have heating rates that are ˜28× and 18× greater, respectively, on a per-weight basis. Heating rates were also found to be power dependent.

FIG. 21B illustrates how the heating rates were determined. Suspensions of m-/s-SWNTs (green and red, respectively) were exposed to the RF field and recorded using an IR camera to give a graph of temperature versus time. This process was then repeated for filtered samples to measure the supernatant heating rates, which were almost identical to the Pluronic aqueous solution, indicating no sample contaminants. Using a linear function to model the heating curves, the heating rates could then be extracted.

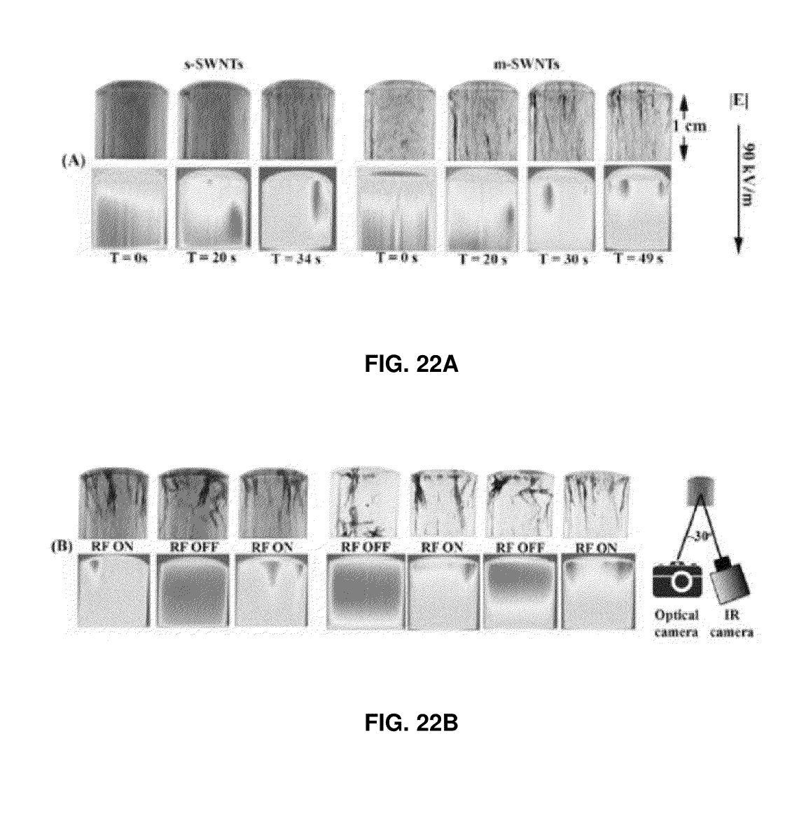

Applicants also noted that m-/s-SWNTs would align and aggregate (within the suspensions) in response to the RF field, with the direction of alignment being parallel to the RF electric-field vector. This is highlighted in FIG. 22A. For aqueous m-/s-SWNT samples (no NaCl), both types of SWNTs would eventually form long (˜1 cm), highly aligned, vertical SWNT-ropes in the solution. In addition, by looking at the IR thermal videos (not shown), the majority of heat production was generated from these SWNT-ropes. By turning the RF field on-and-off (FIG. 22B), these SWNT-ropes alternate between forming and collapsing.

Although both SWNT types heat greater than AuNPs on a per-weight basis, the effect of total surface area was also compared, as this was deemed a pertinent factor in AuNP heating mechanisms. The surface area of a SWNT (SASWNT) can be modeled using the standard equation for a cylinder, 2πrl, where r is the nanotube radius (˜0.5 nm) and l is the SWNT length (300 nm-2 μm). Equation 8 can be used to calculate the weight of each SWNT.

W_{SWNT}=1/1315πld(grams) (8)

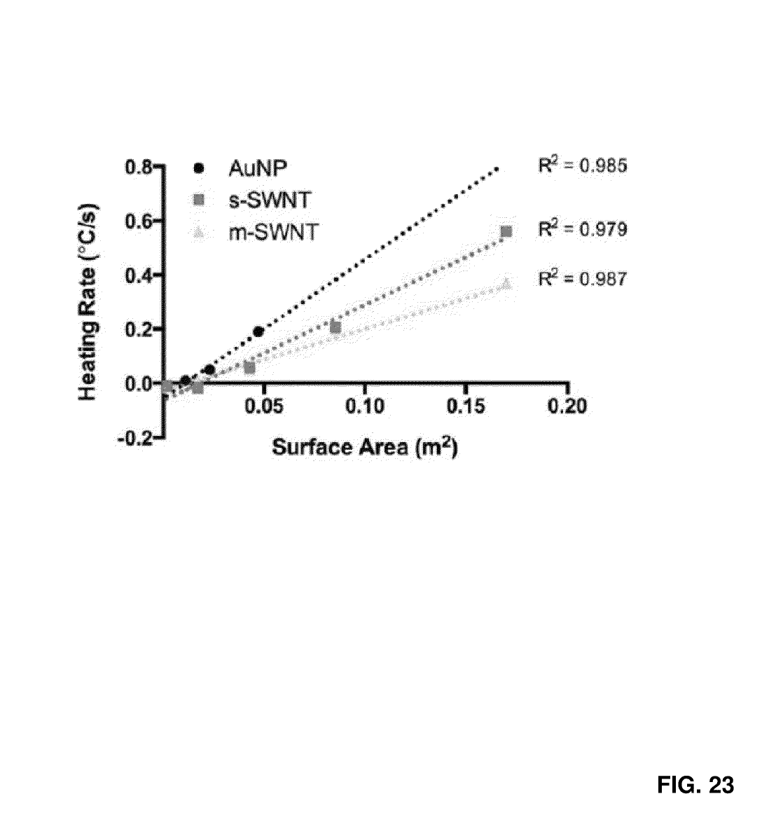

In Equation 8, d is the SWNT diameter. Using data from previous work (The Journal of Physical Chemistry C 2012, 116, 24380-24389), Applicants plotted (FIG. 23) the heating rates of AuNPs and m-/s-SWNTs as a function of total surface area in a 1.3 ml sample solution. By observing the linear regression curves, it can be seen that AuNPs would actually have greater heating rates than m-/s-SWNTs in regards to total surface area. This could be both due to the large negative charge of the AuNP citrate-capping agent as well as the possible passivation of the m-/s-SWNT surface charge due to the Pluronic F108 surfactant. Also, the concentration of AuNPs needed to achieve these heating rates would be ˜10 k mg/L, which cannot be reproduced in the lab without aggregation and was shown to negate RF-induced heating.

### Example 3.6

### SWNT Heating Rates in NaCl Solutions

Using the highest SWNT concentration of 100 mg/L, Applicants then sought to investigate SWNT heating rates versus host medium conductivity, which was modulated using NaCl solutions. Aqueous NaCl solutions (all with 2% w/v Pluronic F108) across the molarity range 10⁴ to 10³ mM were combined with m-/s-SWNTs at a concentration of 100 mg/L. These were then exposed to the RF field and filtered to remove the supernatant, which was then re-exposed.

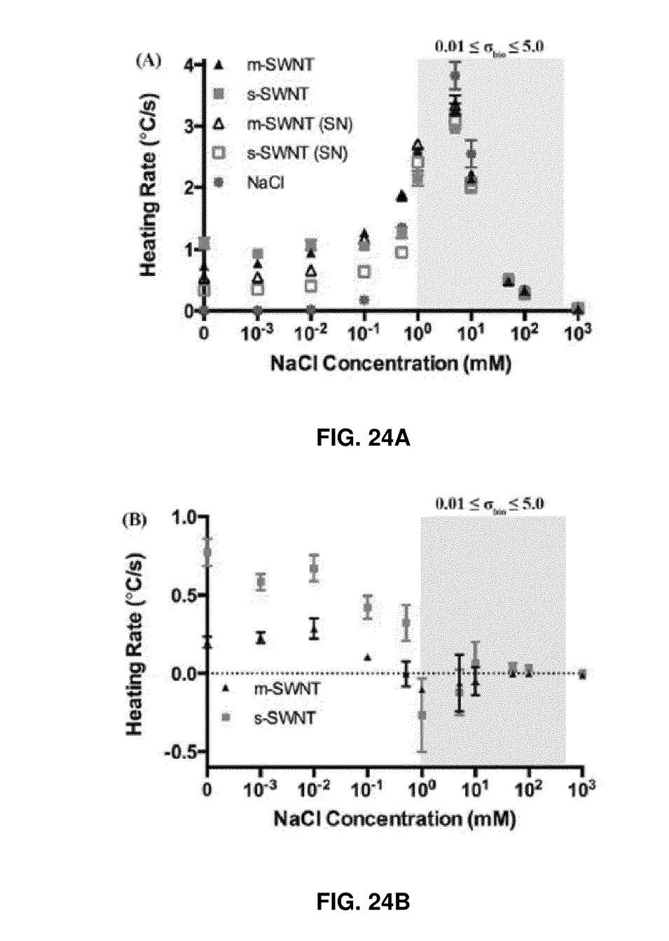

FIG. 24A depicts the heating rates of the SWNT suspensions as a function of NaCl concentration. For clarity, FIG. 24B shows the subtraction of the supernatant heating rates from the m-/s-SWNT suspensions. For NaCl molarities of less than 1 mM (where heating effects were observable), a concentration dependence was also evident. Note the shaded area of biological relevance as these NaCl concentrations (1 to 500 mM) mimic typical biologic conductivity (σbio) ranges of 0.01 to 5.0 S/m, which would come into effect if these results were to be applied to areas such as radiofrequency cancer therapy.

For molarities of more than 1 mM, it is clear that heating is dominated by the rapid bulk ionic heating of the NaCl solutions themselves with maximum heating occurring at 5 mM NaCl concentration. This ‘bell-shaped’ heating effect has also been seen in other RF systems at the same frequency and is the result of the electric field being greatly affected by the dielectric properties of the solutions and the boundary conditions. The effect of the sample holder geometry can also influence this heating rate trend.

As shown in FIG. 24B, there is a slight increase in heat production for the m-SWNT solutions. Moreover, the s-SWNT heating rates are almost 3-4 times greater and are inversely proportional to host conductivity.

### Example 3.7

### Prediction of Heating Rates from Permittivity

A comprehensive analysis of the dielectric properties of saline solutions containing varying concentrations of SWNTs has previously been documented, where the complex permittivity is provided by Equation 9.

∈ₕ=∈′(ω)−i∈″(ω) (9)

In Equation 9, ω is the radial frequency (rad/s), ∈′ and ∈″ represent the real and imaginary components of the permittivity, respectively, and are directly related to the amount of energy stored in a medium (∈′) and the amount of energy dissipated as heat (∈″). The imaginary component of the complex permittivity can be used to define RF power absorbed per unit mass of dielectric material (W/Kg) through the specific absorption rate (SAR), as demonstrated in Equation 10.

$$SAR = \frac{{ɛ}_{0} {ωɛ}^{″}}{ρ} \qquad ( 10 )$$

Therefore, analysis of the complex permittivity can directly lead to predictions and interpretation of RF-induced heating in dielectric materials, such as SWNTs in saline. For instance, direct measurements of the complex permittivity's of colloids containing SWNTs in normal saline (0.9% w/v of NaCl) across the frequency range of 20 MHz to 1 GHz have been performed. Their SWNT concentrations ranged from 111.6 mg/L to 1116 mg/L and were suspended in the saline solutions using a 0.02% Pluronic F108 surfactant to prevent SWNT flocculation. The results demonstrated that the concentrations of SWNTs in saline solutions caused a linear rise in their electrical conductivities and that the SAR and heating of the solutions under RF irradiation should therefore also increase in a linear manner.

The dielectric properties of NaCl solutions (∈h) can be described by the Cole-Cole relaxation function, as demonstrated in Equation 11.

$${ɛ}_{h} ( ω ) = {ɛ}^{′ ( ∞ )} + \frac{{ɛ}^{′ ( 0 )} - {ɛ}^{′} ( ∞ )}{1 - {( {iωτ}_{w} )}^{1 - α}} + i \frac{{σ}_{saline}}{{ωɛ}_{0}} \qquad ( 11 )$$

In Equation 11, ∈′(0) and ∈′(∞) are the values of ∈′ at frequency limits of 0 and ∞ Hz, respectively. Likewise, τw is the dielectric relaxation (or decay) of the solvent, σs is the static conductivity, and a is the relaxation distribution parameter. Equation 11 can also be integrated into the Waterman-Truell formula to estimate the relative permittivity of SWNT suspensions (∈eff (ω)), as shown in Equation 12.

$${ɛ}_{eff} ( ω ) = {ɛ}_{h} ( ω ) + \frac{1}{3 {ɛ}_{0}} {\sum }_{j} {\int }_{0}^{∞} {α}_{j} ( ω , L ) {N}_{j} ( L ) d L \qquad ( 12 )$$

In Equation 12, Nj(L) describes the number density of SWNTs of type j, length L and radius Rj, and αj(ω,L) is the axial polarizability of an isolated SWNT of type j. Using Equations 11 and 12, the effective permittivity and relative absorption rates of saline suspensions (0.9% w/v of NaCl) containing 1120 mg/L of SWNTs of both metallic (15,0) and semiconducting (14,0) types, at a metallic to semiconducting density ratio of 1:2 were modeled by others. Variations in tube radius were neglected as they were previously found to have a slight effect on the axial polarizability of metallic tubes. Further, the conductivity of semiconducting tubes of small radius (1-3 nm) across the frequency range of interest (10 MHz-1 GHz) does not depend on chirality and is dominated by impurity doping mechanisms. These inclusions can be extended to, and are valid for, SWNT suspensions of metallic and semiconducting tubes with varying chirality.

The theoretical values matched in accordance with the data generated by others, and they demonstrated a 2-fold absorption enhancement in the SWNT-saline solution at a concentration of 1120 mg/L, which is approximately 10 times higher than the SWNT concentrations used in Applicants' studies. Further, the prior studies concluded that the application of longer SWNTs (1-3 μm) is more effective in enhancing energy dissipation in the solutions than SWNTs of shorter lengths (0.1-0.3 μm).

In terms of molarity, a normal saline solution (0.9% w/v NaCl) is equivalent to a 154 mM NaCl solution. From looking at similar values in FIGS. 24A-B, it can be seen that the heating rates for m-/s-SWNTs in a 100 mM NaCl suspension are all equivalent and no meaningful data can be extracted in regards to SWNTS enhancing the absorption and heating rates of the solutions.

The results demonstrated previously by others indicate that heating would increase linearly across the range of 110 mg/L to 1110 mg/L. However, such observations are not evident in this Example due to the concentration limit of 100 mg/L. Applicants also found that concentrations higher than 100 mg/L would result in heavily aggregated solutions that would eventually precipitate out of solution, thereby making it difficult to differentiate heating mechanisms from individual and aggregated SWNTs.

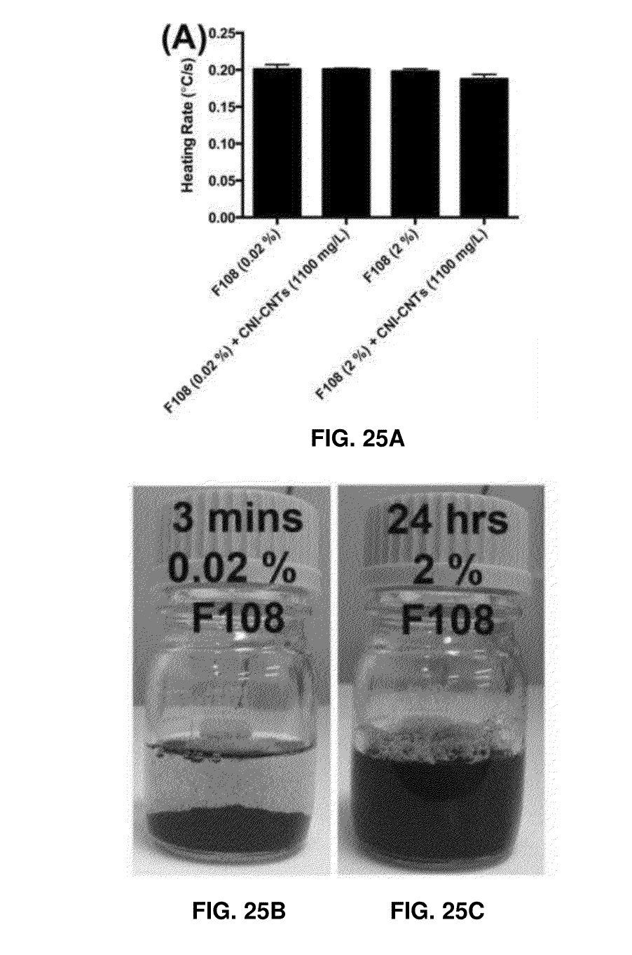

Using another source of SWNTs (Carbon Nanotechnologies, Houston, Tex.), Applicants were able to prepare high concentrations (1100 mg/L) of SWNTs suspended in 0.02% Pluronic in normal saline (154 mM NaCl), which were the experimental and theoretical conditions used by others. As can be seen in FIGS. 25A-C, there is no enhancement observed at all for highly concentrated SWNTs when compared to the non-SWNT solutions. Further, to minimize the effects of aggregation diminishing heat production, Applicants also prepared a highly concentrated SWNT solution in a 2% Pluronic solution—similar to the solutions used through out this Example.

Higher concentrations of Pluronic F108 were deemed necessary, as the SWNTs would drop out of suspension within 3 minutes when using 0.02% Pluronic, compared to being stable for 24 hours in the 2% Pluronic solutions (FIGS. 25B-C, respectively). Throughout Applicants' initial studies, Applicants examined the heating rates of s-SWNTs at a concentration of 300 mg/L suspended in identical conditions (0.02%, 154 mM NaCl). Similar to other results, no enhancements were observed (ESM S11).

In sum, Applicants have investigated the RF-induced heating rates of electronically enriched (95%) m-/s-SWNTs as a function of SWNT concentration and host medium conductivity. Heating rates for s-SWNTs were inversely proportional to host conductivity and were found to increase linearly up to 0.8° C./s, as the host molarity decreased below 1 mM (˜0.01 S/m). Smaller values of heat production from the m-SWNT samples were also visible (˜0.25° C./s) across the same molarity range. Furthermore, both types of SWNT-types aligned and aggregated in suspension to form ‘SWNT-ropes’ of length equal to the vertical dimension of the sample holder (˜1 cm) during heat production, most likely due to dielectrophoretic force and torque phenomena. Infrared (IR) thermal imaging data showed that the heat produced is localized and liberated from the SWNT-ropes much more than the surrounding SWNT solution. For molarities above 1 mM, the bulk heating effects of the solution dominated, obscuring any m-/s-SWNT enhancements. Applicants' results were also compared and evaluated alongside theoretical predictions concerning m-/s-SWNTs in a conductive host. Although there are discrepancies between observed and theorized heating effects, this is most likely due to SWNTs forming aggregated bundles in the NaCl solutions, which effectively diminish any theorized enhancement effects.

What is claimed is:

1. A method of optimizing a treatment efficacy of a biological system, said method comprising:
tuning a property of the biological system,
wherein the tuning comprises adding an optimizing agent to the biological system.

2. The method of claim 1, wherein the biological system is selected from the group consisting of aqueous biological systems, body fluids, blood, solid biological systems, tissues, organs, vasculatures, matrices, and combinations thereof.

3. The method of claim 1, wherein the biological system comprises a tissue.

4. The method of claim 3, wherein the tissue is selected from the group consisting of human tissues, animal tissues, tumors, tumor-containing tissues, natural tissues, synthetic tissues, fatty tissues, non-fatty tissues, and combinations thereof.

5. The method of claim 1, wherein the biological system is part of a subject or isolated from the subject.

6. The method of claim 5, wherein the subject is a human being suffering from a condition.

7. The method of claim 6, wherein the condition is selected from the group consisting of cancer, tumor growth, infectious diseases, inflammatory conditions, and combinations thereof.

8. The method of claim 6, wherein the condition is cancer.

9. The method of claim 1, wherein the adding comprises contacting the biological system with the optimizing agent.

10. The method of claim 9, wherein the contacting comprises direct application of the optimizing agent to the biological system.

11. The method of claim 9, wherein the contacting comprises application of the optimizing agent to a subject containing the biological system.

12. The method of claim 11, wherein the application is selected from the group consisting of oral administration, inhalation, subcutaneous administration, intravenous administration, intraperitoneal administration, intramuscular administration, intrathecal injection, topical administration, and combinations thereof.

13. The method of claim 1, wherein the optimizing agent is selected from the group consisting of kosmotropic materials, non-polar materials, carbon nanotubes, salts, and combinations thereof.

14. The method of claim 1, wherein the optimizing agent comprises a kosmotropic material.

15. The method of claim 14, wherein the kosmotropic material is selected from the group consisting of alcohols, zwitterionic molecules, amines, neutral molecules, water soluble molecules, water soluble nanomaterials, carbohydrates, amino acids, organosulfur compounds, and combinations thereof.

16. The method of claim 14, wherein the kosmotropic material comprises an organosulfur compound.

17. The method of claim 16, wherein the organosulfur compound is dimethyl sulfoxide.

18. The method of claim 14, wherein the kosmotropic material comprises a carbohydrate selected from the group consisting of maltose, glucose, sucrose, trehalose, and combinations thereof.

19. The method of claim 14, wherein the kosmotropic material comprises an alcohol selected from the group consisting of methanol, glycerol, polyhydric alcohols, sorbitol, glycols, ethylene glycol, propylene glycol, and combinations thereof.

20. The method of claim 14, wherein the kosmotropic material comprises a water soluble nanomaterial selected from the group consisting of functionalized fullerenes, functionalized carbon nanotubes, shortened carbon nanotubes, derivatized nanoparticles, and combinations thereof.

21. The method of claim 14, wherein the kosmotropic material comprises an amine selected from the group consisting of zwitterionic amines, betaines, glycine, methylated glycine, proline, sarcosine, N,N-dimethyl glycine, betaines, glycine betaine, proline betaine, trimethylamine oxide, and combinations thereof.

22. The method of claim 1, wherein the optimizing agent comprises non-polar materials.

23. The method of claim 22, wherein the non-polar materials are selected from the group consisting of lipids, oils, fats, porphyrins, aromatic compounds, ethiodol, and combinations thereof.

24. The method of claim 1, wherein the optimizing agent comprises carbon nanotubes.

25. The method of claim 24, wherein the carbon nanotubes are selected from the group consisting of single-walled carbon nanotubes, multi-walled carbon nanotubes, metallic carbon nanotubes, semiconducting carbon nanotubes, and combinations thereof.

26. The method of claim 1, wherein the optimizing agent comprises a salt.

27. The method of claim 26, wherein the salt is selected from the group consisting of inorganic salts, metallic salt solutions, non-metallic salt solutions, electrolyte solutions, monovalent salts, divalent salts, trivalent salts, and combinations thereof.

28. The method of claim 1, wherein the property is selected from the group consisting of dielectric properties of the biological system, physical properties of the biological system, the heating rate of the biological system, the heat capacity of the biological system, the conductivity of the biological system, the permittivity of the biological system, the density of the biological system, the ionic mobility through the biological system, and combinations thereof.

29. The method of claim 1, wherein the property comprises the heating rate of the biological system.

30. The method of claim 1, wherein the property comprises the heating rate of the biological system exposed to a radiofrequency (RF) field.

31. The method of claim 1, wherein the tuning increases the heating rate of the biological system.

32. The method of claim 1, wherein the tuning reduces the conductivity of the biological system.

33. The method of claim 1, wherein the tuning decreases the permittivity of the biological system.

34. The method of claim 1, wherein the tuning reduces the ionic mobility through the biological system.

35. The method of claim 1, wherein the tuning comprises:
(a) determining a property parameter of the biological system;
(b) selecting the optimizing agent to be added to the biological system based on the determined property parameter; and
(c) adding the optimizing agent to the biological system.

36. The method of claim 35, wherein the determining comprises measuring the property parameter.

37. The method of claim 35, wherein the determining comprises estimating the property parameter.

38. The method of claim 35, wherein the adding comprises contacting the biological system with the optimizing agent.

39. The method of claim 1, wherein the property of the biological system is tuned in a selective manner.

40. The method of claim 39, wherein the property of a first biological system is tuned while the property of a second biological system remains substantially the same.

41. The method of claim 40, wherein the first biological system is associated with a condition, and wherein the second biological system is not associated with the condition.

42. The method of claim 40, wherein the first biological system is a tumor, and wherein the second biological system is normal tissue.

43. The method of claim 1, wherein the treatment comprises radiation treatment of the biological system.

44. The method of claim 1, wherein the treatment comprises heat treatment of the biological system.

45. The method of claim 1, wherein the treatment occurs in a radiofrequency field.

46. The method of claim 1, further comprising a step of treating the biological system.

47. The method of claim 46, wherein the treating comprises exposing the biological system to heat.

48. A method of optimizing a treatment efficacy of a biological system, said method comprising:
(a) determining a property parameter of the biological system;
(b) selecting an optimizing agent to be added to the biological system based on the determined property parameter; and
(c) adding the optimizing agent to the biological system,
wherein the optimizing agent comprises a kosmotropic material.

49. The method of claim 48, wherein the biological system comprises a tissue.

50. The method of claim 48, wherein the adding comprises contacting the biological system with the optimizing agent.

51. The method of claim 48, wherein the kosmotropic material is selected from the group consisting of alcohols, zwitterionic molecules, amines, neutral molecules, water soluble molecules, water soluble nanomaterials, carbohydrates, amino acids, organosulfur compounds, and combinations thereof.

52. The method of claim 48, wherein the property comprises the heating rate of the biological system.

53. The method of claim 48, further comprising a step of treating the biological system.

54. The method of claim 53, wherein the treating comprises exposing the biological system to heat.
